(19) United States
(12) Patent Application Publication
(10) Pub. No.: US 2007/0190155 A1
(43) Pub. Date: Aug. 16, 2007

(54) Molecular programming of nanoparticle systems for an ordered and controlled sequence of events for gene-drug delivery

(76) Inventors: James F. Leary, West Lafayette, IN (US); Tarl W. Prow, Baltimore, MD (US)

Correspondence Address: LOCKE LIDDELL & SAPP LLP;ATTN: IP DOCKETING, 600 TRAVIS, 3400 CHASE TOWER, HOUSTON, TX, 77002-3095, US

(21) Appl. No.: 10/591,213

(22) PCT Filed: Mar. 4, 2005
(86) PCT No.: PCT/US05/06692

§ 371 Date: Aug. 31, 2006

Related U.S. Application Data
(60) Provisional application No. 60/550,816, filed on Mar. 5, 2004.

(51) Int. Cl. A61K 48/00; A61K 9/14

(52) U.S. Cl. 424/489; 514/44; 977/906

(57) ABSTRACT

The disclosure provides a nanodelivery system and related process having complex, multilayered nanoparticles for sophisticated drug/gene delivery systems to intracellular portions of a cell. Outermost layers can include cell targeting and cell-entry facilitating molecules. The next layer can include intracellular targeting molecules for precise delivery of the nanoparticle complex inside the cell of interest. Molecular biosensors can be used to confirm the presence of expected molecules as a surrogate molecule for signs of infection, for activation in radiation damage, or other criteria, prior to delivery of counter-measure molecules such as drugs or gene therapy. The biosensors can also be used as a feedback control mechanism to control the proper amount of drug/gene delivery for each cell. Further, the nanodelivery system can be used to restrict any cells from encountering the drug unless that cell is specifically targeted. Successful targeting can be verified by 3D multispectral confocal microscopy.

This application claims the benefit of U.S. Provisional Application Ser. No. 60/550,816, filed Mar. 5, 2004 and incorporates by reference such provisional application in its entirety herein.

This work was supported by the Biomolecular, Physics and Chemistry Program under NASA grant NAS2-02059.

### FIELD

The invention relates to medicinal delivery systems and methods. More specifically, the invention relates to subcellular drug and gene delivery systems and methods.

### BACKGROUND

A goal of modern medicine is to provide earlier diagnostics, so that diseases can be treated when they are most treatable. Dramatic results have been achieved in a number of diseases. However, diagnostics and therapeutics have not yet been combined in a system to not only diagnose, but also to treat, at the earliest possible stage—perhaps before actual symptoms appear. For example, the three conventional treatments for cancer are (1) surgical removal of the tumor, (2) radiation therapy, and (3) chemotherapy. These treatments occur necessarily after the cancer has been exhibited on a sufficiently large scale to allow detection. Further, conventional medicine is not readily available to much of humanity because it is labor-intensive, sophisticated, and expensive.

Some recent medical breakthroughs to avoid some of the conventional treatments have occurred through gene delivery systems. However, existing gene delivery systems have a variety of limitations (De Smedt, Demeester et al. 2000). These systems are designed to eliminate an infection by transferring a therapeutic gene to host cells. However, they have been are largely unsuccessful, because only low doses of genetic material can reach the specific cell types that are infected. Increased side effects also include the non-specific targeting and treatment of non-infected cells with high levels of genes and the host cells reacting to the carrier molecules associated with their delivery. To date, the available gene delivery systems reported have been those that contain retroviral vectors, are liposome based, or are systems in which naked DNA, RNA and modified RNA have been injected directly into the blood stream, all of which produce many undesirable side effects that can compromise the treatment of patients. Retroviral vectors have potential dangerous side effects which include incorporation of the virus into the hosts immune system and hence, have been less successful than originally hoped (De Smedt, Demeester et al. 2000). Liposome based gene transfer has relatively low transfection rates, are difficult to produce in a specific size range, can be unstable in the blood stream, and are difficult to target to specific tissues (De Smedt, Demeester et al. 2000). Injection of naked DNA, RNA, and modified RNA directly into the blood stream leads to clearance of the injected nucleic acids with minimal beneficial outcome (Sandberg, Sproul et al. 2000). As such, there is currently a need for a gene delivery system which has minimal side effects but high affectivity and efficiency. One such system could be that of the self-assembled nanoparticles coated with targeting biomolecules (Lvov and Caruso 2001).

The broad concept of nanotechnology encompasses the concept of “nanomedicine,” a fundamentally different paradigm for medicine. It is “nano” not only because it uses nanometer scaled tools, but also because it employs a cell-by-cell regenerative and repair philosophy working at the single cell level rather than at the organ level.

To date, the majority of nanoparticle research has been carried out by materials scientists, but recent trends have brought these tools into the hands of biologists. Nanoparticles have found two broad niches in biology, detection technologies and payload delivery (Koropchak, et al., 1999; Douglas, et al., 1987). Generally, current nano-based system have only gone as far as use of initial targeting molecules on nanoparticles and further refinements in encapsulation of drugs or genes. Since the late 1970s, nanoparticles have been used to deliver drugs (Douglas, et al., 1987; Kreuter, et al., 1979). The majority of publications concerning biological applications of nanoparticles are focused on the delivery of chemotherapeutic agents with nanoparticles ranging from 2 to 3000 nm. Nanoparticle mediated gene delivery has recently emerged as a promising tool for gene therapy strategies (Panyam, et al., 2003; Vijayanathan et al., 2002; Benns et al., 2000). However, among other issues, one of the main problems with using nanoparticles for gene delivery is the construction, cost, and quality control of the nanoparticles themselves. Further, there needs to be a commercially available nanoparticle that is readily available, easy to use, and flexible.

Besides known diseases needing treatment, other needs are becoming apparent, especially in extreme environments and/or extreme circumstances. For example, NASA is seeking solutions as part of their nanomedicine for astronauts in longer voyage space exploration. For example, on a manned roundtrip voyage to Mars and exploration on the surface of that planet, astronauts will encounter levels of radiation that are impossible to shield. There will also be no hospitals with either diagnostic or therapeutic treatments. The signal delays in Earth-Mars communications represents a major challenge to telemedicine, and largely precludes procedures requiring real-time Earth control. Any systems brought along must be small, low weight, intelligent, and autonomous. While there may be large radiation damage effects, the most likely scenario is a series of fractionated radiation doses, each of which is not necessarily a pivotal event, but whose accumulation can lead to further downstream events such as organ injury or cancer.

Thus, there remains a need to an improved nanoparticle system that can be used for medicinal purposes.

### SUMMARY OF THE INVENTION

The disclosure provides a nanodelivery system and related process having complex, multilayered nanoparticles for sophisticated drug/gene delivery systems to intracellular portions of a cell. Outermost layers can include cell targeting and cell-entry facilitating molecules. The next layer can include intracellular targeting molecules for precise delivery of the nanoparticle complex inside the cell of interest. Molecular biosensors can be used to confirm the presence of expected molecules as a surrogate molecule for signs of infection, for activation in radiation damage, or other criteria, prior to delivery of counter-measure molecules such as drugs or gene therapy. The biosensors can also be used as a feedback control mechanism to control the proper amount of drug/gene delivery for each cell. Further, the nanodelivery system can be used to restrict any cells from encountering the drug unless that cell is specifically targeted. Successful targeting can be verified by 3D multispectral confocal microscopy. These single cell molecular morphology measurements can be extended from individual cells, to other cells in a tissue in tissue monolayers or tissue sections.

The purpose of this disclosure is to produce multifunctional and multi-step nanoparticle systems that follow a predictable and well defined sequence of events (“molecular programming”) as laid out by a molecular chain of events and can be applied to the delivery of drugs, genes, or other medicinal purposes. These events include, but are not limited to, events such as initial cell targeting, facilitation of cell entry, intracellular re-targeting, intracellular anchoring to the site of drug/gene delivery, drug or gene delivery, controlled delivery of the drugs or genes within single cells through feedback loops facilitated by molecular biosensors and other molecules, or a combination thereof. In some embodiments, magnetic nanoparticles can be used. The present disclosure can provide a strategy of nanomedicine to repair the radiation damage on a continuous basis using DNA repair enzymes in nanoparticle systems targeted to cells likely to have experienced radiation as a counter-measure to more serious radiation injury at the organ level. Further, the same or similar strategy can be used to treat numerous other diseases and infirmities.

The disclosure provides in at least one embodiment a multifunctional and multi-step nanodelivery system and related method consisting of targeting molecules, entry facilitating molecules, re-targeting molecules, anchoring molecules, drugs or genes that are either driven or controlled through molecular biosensor feedback loops for controlled drug-gene delivery.

The disclosure provides also provides a nanodelivery system and related method with molecular error-checking based on desired or permissible Boolean logic conditions (based on presence or absence of specific molecules on or within the cell) to reduce false positive targeting which then lowers undesired, adverse bystander side reactions.

The disclosure provides a nanosystem and related method having molecules that use, create or reorganize molecules already within individual living cells such that a single nanoparticle can manufacture enough drugs or genes to have a therapeutic response. This solves the problem of how to deliver enough drugs or genes to single cells in-vivo to achieve therapeutic value.

The disclosure provides a nanosystem and related method that positions itself at the active site of importance for subsequent drug-gene delivery within a single living cell through the use of localization or anchoring sequences

The disclosure provides a multi-functional and multi-step nanodelivery system having one or more nanoparticles, the nanoparticles comprising: one or more targeting molecules adapted to target the nanoparticle to one or more cells; one or more cellular entry facilitating molecules coupled to the targeting molecules; one or more anchoring molecules coupled to the entry facilitating molecules; one or more drugs or genes coupled to the anchoring molecules; and one or more molecular biosensors adapted to control an intracellular delivery of a quantity of the drugs or genes in a feedback loop at a single cell level.

The disclosure also provides a nanodelivery system having two or more nanoparticles for delivery of a drug or gene to an intracellular location, comprising: a first nanoparticle having a first targeting molecule and a first component of a drug or gene; a second nanoparticle having a second targeting molecule and a second component of a drug or gene different than the first component; wherein the drug or gene is adapted to provide a desired reaction upon a successful targeting of the first and second targeting molecules and a combination of the first and second components of the drug or gene.

The disclosure further provides a multi-functional and multi-step nanodelivery system having one or more nanoparticles, the nanoparticles comprising: one or more targeting molecules adapted to target the nanoparticle to one or more cells; and one or more cellular entry facilitating molecules coupled to the targeting molecules; the nanoparticle being adapted to manufacture quantities of the desired drug or gene from an intracellular location of the cell using one or more intracellular native components.

The disclosure provides a multi-functional and multi-step nanodelivery system having one or more nanoparticles, the nanoparticles comprising: one or more targeting molecules adapted to target the nanoparticle to one or more cells; and one or more cellular entry facilitating molecules coupled to the targeting molecules; and one or more anchoring molecules coupled to the entry facilitating molecules and adapted to locate at least one nanoparticle at an intracellular selected site for subsequent intracellular drug or gene delivery.

The disclosure further provides a process for producing a multi-functional and multi-step nanodelivery system, acting in an autonomous controlled sequence of events at the molecular level, comprising: obtaining a nanoparticle; coupling a drug or gene to the nanoparticle; coupling a molecular biosensor to the drug or gene; and coupling a cell targeting molecule to the molecular biosensor.

### BRIEF DESCRIPTION OF THE DRAWINGS

A more particular description, briefly summarized above, may be had by reference to the embodiments illustrated in the appended drawings, forming part of the present specification and described herein. It is to be noted, however, that the appended drawings illustrate only some embodiments described herein and are therefore not to be considered limiting of the disclosure's scope, in that there can be other equally effective embodiments.

FIG. 1A is a cross-sectional schematic diagram of one embodiment of a multilayered nanomedicine system.

FIG. 1B is a schematic diagram of a method of construction of a nanomedicine delivery system.

FIGS. 1C-1F are representations of intracellular targeted delivery, where FIG. 1C is a representation of a cell with an uncoated nanoparticle, FIG. 1D is a representation of a cell with a nanoparticle coated with HIV tat fragment, FIG. 1E is a representation of a cell with a nanoparticle coated with anti-CD95, and FIG. 1F is a representation of a cell with a nanoparticle coated with a 6× Arginine peptide.

FIG. 2 is a schematic illustrating a spectral unmixing algorithm implemented on a multispectral confocal microscope.

FIG. 3A is a representation of a photomicrograph of a conventional antibody labeling system.

FIG. 3B is a representation of a photomicrograph of a nanoparticle antibody labeling system.

FIG. 4A shows a 10× objective phase-fluorescence photomicrograph of the combined mixture of cells and nanoparticles.

FIG. 4B shows a 40× two color fluorescence only photomicrograph

FIG. 5 is a schematic diagram and sequence of one embodiment of a molecular biosensor.

FIGS. 6A, 6B, 6C are representations of photomicrographs showing results from the construct of FIG. 5.

FIGS. 7A, 7B, 7C, 7D, 7E, 7F represent photomicrographs of intracellular co-localization of molecular biosensors.

FIG. 8 is a schematic diagram of the transcription of the DNA repair enzymes initiated by ARE complex binding.

FIG. 9A is a schematic representing a progression of ROS treated cells.

FIGS. 9B, 9C represent photomicrographs of the cells with an ROS sensor and without a sensor, respectively.

FIG. 10A is representation of a photomicrograph of results of a DNA repair enzyme with no localization anchoring sequence.

FIG. 10B is a representation of a photomicrograph of results of a DNA repair enzyme with a mitochondrial localization anchoring sequence with transient expression.

FIG. 10B is a representation of a photomicrograph of results of a DNA repair enzyme with a mitochondrial localization anchoring sequence, with stable expression.

FIG. 11 is a schematic representation of a conjugation of DNA to a magnetic particle (MN).

FIG. 12 is a schematic representation of a purification of DNA tethered MN.

FIG. 13 is a schematic representation of expression levels of EGFP from DNA tethered MN.

FIG. 14 is a schematic representation of an expression of EGFP from MN tethered to EGFP DNA.

FIG. 15 is a schematic representation of recovery and PCR of DNA tethered to magnetic nanoparticles.

FIG. 16 is a schematic representation of magnetic nanoparticle delivery of genes in vivo.

FIG. 17A is a schematic representation of a virus infecting a cell.

FIG. 17B is a schematic representation of a nanoparticle construct having a virus appearance with an antiviral payload.

FIG. 18 is a schematic representation of production of a therapeutic gene.

FIG. 18A is a schematic representation of transcribed gene being cleaved.

FIG. 18B is a schematic representation of the therapeutic gene of FIG. 18A bound to a genome.

FIG. 18C is a schematic representation of the internal ribosome entry site (IRES) cleaved by the therapeutic gene of FIG. 18B.

FIGS. 19A-19D are a representation of a single cell ribozyme therapy, showing the results with control data, such that FIG. 19A represents cells with stained DNA, FIG. 19B represents cells stained with a molecular biosensor, FIG. 19C represents a therapeutic response, and FIG. 19D represents a composite image.

### DETAILED DESCRIPTION

In general, this disclosure provides a nanomedical system and method that can be used for diagnostics, therapeutics, or a combination thereof by use of a multilayered nanoparticle system. The multilayered nanoparticle system can built on a nanoparticle core of polystyrene, silica, gold, iron, or other material. Drug or genes, molecular biosensors, and extracellular (as well as intracellular) targeting molecules can be added to the nanoparticles to construct a “nanomedical system”.

The disclosure provides a next generation nanomedicine technology for continuous and linked molecular diagnostics and/or therapeutics (“theragnostics”). The nanomedicine system can provide “sentinel” nanoparticles that can seek out diseased (e.g. cancerous) cells, enter those living cells, and either perform repairs or, if necessary, induce those cells to die through apoptosis. These nanoparticles are envisioned as multifunctional, autonomous “smart drug/gene delivery systems.”

Conventional nanotechnology using nanoparticles for medicinal purposes attempts to target the entire cell and deliver a treatment to that cell or even that region. What has been lacking heretofore, however, is that such a process is only a first stage of a multi-stage process of drug-gene delivery, which generally according to the teaching of this disclosure, needs to occur in a controlled sequence to be successful and/or effective. Merely delivering the drug/gene to a cell surface by conventional targeting does not insure that it is delivered to the site of required action within the cell. There generally needs to also be a multi-step intracellular molecular targeting process. The nanosystems can contain intracellular targeted molecules that re-direct the nanomedical system to the correct intracellular location for specific molecular and biochemical actions. For example, the interior of a 10 micron diameter cell is approximately a billion times larger than the volume of an individual 10 nm diameter nanoparticle. The present disclosure advantageously provides, among other things, a further step of intracellular targeting is really required to be effective in this process. Three cellular entry facilitation methods, among others, include use of: (i) arginine-repeat peptides, (ii) Lipofectamine™ coatings to promote fusion of nanoparticles with the cell membrane, and (iii) artificial tat-specific sequences, the entry and nuclear targeting molecule used by HIV-1. Using confocal microscopy can insure that the drug/gene is targeted to the correct location within single cells.

Nanoparticle targeting can be accomplished in a variety of ways. Two methods common are use of antibodies (e.g., anti-CD95 antibody) bound to the nanoparticle outer surface, or coating of the outer surface of the nanoparticles with molecules that are the ligands for cellular receptors (e.g., mannose to target nanoparticles to liver cells which have mannose receptors). While antibody targeting is used for in-vitro applications, their use in-vivo can be problematical since some of these targeting antibodies can illicit an immune response from the human or animal. The mannose represents less of a problem in this regard, because the body already recognizes mannose and does not tend to mount an immune response against it.

The other significant difference between in-vitro and in-vivo targeting is the great difference in specificity required. Cells are usually not rare in-vitro, while targeted cells are almost always rare in-vivo. Rare cell targeting presents considerable challenges in terms of specificity. Considering the number of possible interactions in-vivo, the specificity of the overall targeting system should, in most cases, be better than a million to one. No known antibodies alone have this degree of specificity. To solve this problem, Boolean combinations of antibodies are advantageously chosen, as explained below.

Hyperspectral confocal microscopy, described below in reference to FIG. 2, allows a more sensitive detection of nanoparticles within an overlapping, but different, autofluorescence background of the cell. This analysis can be used to validate the interactions of the nanodelivery system and the cellular and intracellular selected components.

The nanosystems of the disclosure generally are multilayered nanoparticles (nanocrystals, nanocapsules) containing cell targeting molecules, intracellular re-targeting molecules, molecular biosensor molecules, and drugs/enzymes/gene therapy. In some embodiments, these “nanomedicine systems” can be autonomous, much like present-day vaccines, but can have sophisticated targeting, sensing, and feedback control systems—much more sophisticated than conventional antibody-based therapies. The fundamental concept of nanomedicine is to not to just kill all aberrant cells by surgery, radiation therapy, or chemotherapy. Rather it is a form of regenerative medicine—to fix cells, when appropriate, one cell-at-a-time, and to preserve and re-build organ systems as “nanosurgery”, while avoiding the effects of a inflammatory response by the body's immune system which in many cases can be more severe than the original disease itself. When cells should not be fixed, such as in cases where an improperly repaired cell might give rise to cancer cells, the nanomedical therapy would be to induce apoptosis in those cells to eliminate them without the damaging bystander effects of the inflammatory immune response system reacting to necrotic cells or those which have died from trauma or injury.

Nanomedicine can be more preventive, combining very early diagnostics with initial therapeutics—the combination which is now referred to as “theragnostics”. Nanomedicine attempts to make smart decisions to either remove specific cells by induced apoptosis or repair them. Single cell treatments can be based on molecular biosensor information that controls subsequent drug delivery to that single cell. Such a system generally could be semi-autonomous, with pre-determined decisions points for when a diagnosed condition warrants treatment. Clinical decisions are usually much delayed by the negative potential side effects of that treatment, particularly if the diagnosis is initially incorrect. The present disclosure is believed to provide an increase in accuracy of treatment and the minimization of unfavorable side effects and bystander effects, resulting in faster autonomous treatments in continuous response to in-vivo molecular diagnostics.

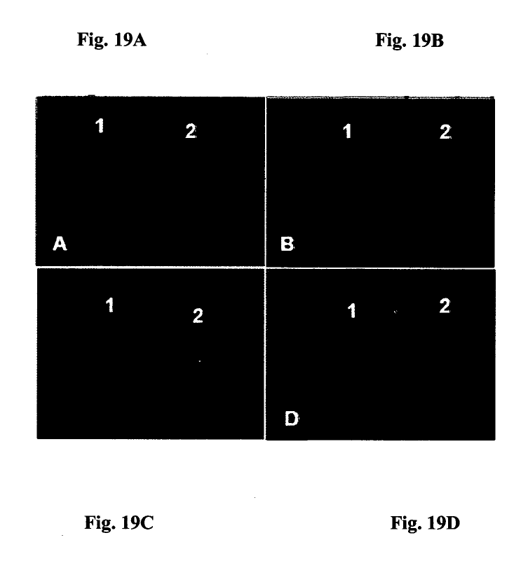

The drugs or genes associated with the nanomedicine systems need to survive the different intracellular microenvironments on the way to their site of action within the cell and be guided by physical or molecular mechanisms to the site of action within a cell. Since delivery of a drug to a cell in-vivo is a “rare-event”, it is advantageous to include error-checking within the system in addition to the molecular targeting, because of the inherent difficulty in not making mistakes in targeting. In order to perform a controlled drug or gene delivery inside single living cells, the present disclosure also provides a feedback control in the drug-gene delivery system. One way to accomplish this feedback control is through the use of molecular biosensors which are connected to the nanosystems. These biosensors can not only sense targets, but can also respond in feedback loops to the relative amount of cellular molecular responses to the drug or gene delivery. In general, molecular biosensors can confirm the correct targeting by sensing the environment and to provide an initial control of drug/gene delivery. In addition, when the drug/gene treatment has resulted in the elimination of the pathogen, there will be a subsequent reduction of the indicator molecules. That this concept works and has been reduced to practice for at least one application as actually shown in reference to FIGS. 19A-19D below. This reduction in indicator molecules can shut off expression of the therapeutic gene due to increased biosensor activity. These secondary checks for correct targeted and control of drug/gene delivery can be used to help minimize “bystander” effects now common as “side effects” in conventional drug administrations.

Since it is difficult to deliver enough drug or gene therapy contained in or on the nanoparticle, and to avoid the effects of “quantized” drug delivery from one, two, three, etc. nanoparticle deliveries per cell, the disclosure provides a system and method to send nanoparticles containing the molecular machinery (e.g. gene sequences that can be transcribed by the host cell or enzymes) for reorganizing or synthesizing therapeutic molecules from raw materials within the cells—effectively setting up a “nanofactory” for manufacturing therapeutic gene sequences under the control of an upstream molecular biosensor that can produce the amount of therapeutic gene necessary, generally no more no less, for successful treatment of that particular single cell.

Integrated Nanoparticle System

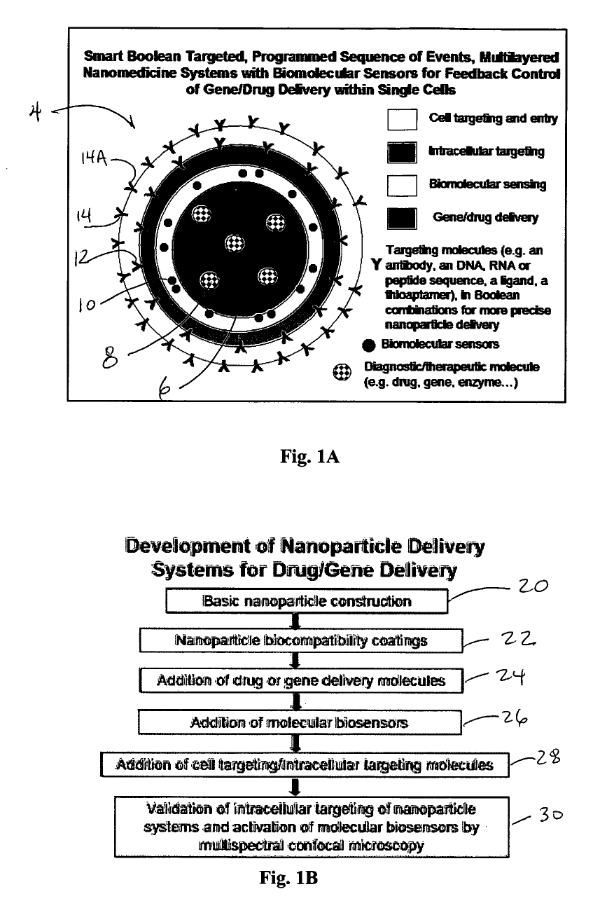

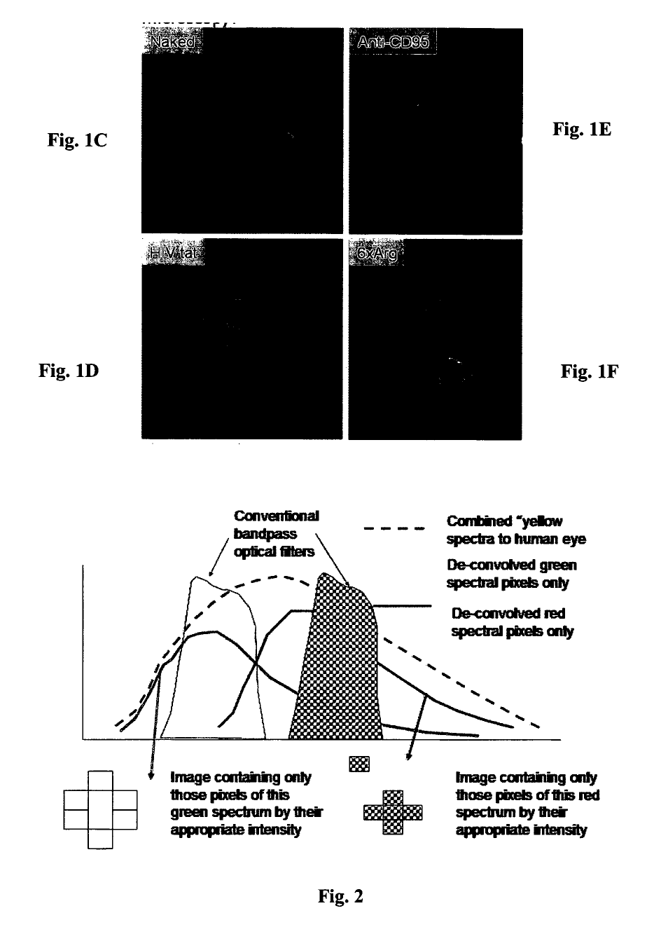

FIG. 1A is a cross-sectional schematic diagram of one embodiment of a multilayered nanomedicine system. FIG. 1B is a schematic diagram of a method of construction of a nanomedicine delivery system. The figures will be described below in conjunction with each other.

Multilayered nanoparticle systems can be constructed which combine cell targeting molecules (e.g. antibodies), membrane entry facilitating molecules, intracellular targeting molecules (e.g. amino acid sequences that target to intracellular organelles), molecular biosensors, and drugs or genes for therapy. Such an integrated sequence of events constitutes an integrated nanodelivery system 4, as shown in FIG. 1A, which can produce a sequence of molecular events. In at least one embodiment, a nanoparticle 6 is used, which can be the nanocrystal, nanocapsules, described above, or other nanoparticle types and portions. A diagnostic and/or therapeutic molecule 8, such as a drug, gene (including an enzyme for present purposes) can be coupled to the nanoparticle 6. The term “coupled,” “coupling,” and like terms are used broadly herein and can include any method or device for securing, binding, bonding, fastening, attaching, joining, inserting therein, forming thereon or therein, communicating, or otherwise associating, for example, biologically, mechanically, magnetically, electrically, chemically, directly or indirectly with intermediate elements, one or more pieces of members together and can further include integrally forming one functional member with another. The coupling can occur in any direction, including rotationally.

A molecular biosensor 10 can be coupled to the molecule 8. The biosensor 10 can be used to control the delivery, expression, manufacture, or other aspects of the molecule 8. In at least one embodiment, the biosensor is “upstream” of the molecule 8 so that the biosensor can activate and deactivate the molecule 8 based on input to the biosensor of predetermined conditions present in the cell or intracellular cell components. An intracellular targeting molecule 12 can be coupled to the biosensor 10. The intracellular molecule (also referred to herein as a “re-targeting”) molecule can provide additional targeting the nanodelivery system after the system has entered the cell.

A cell targeting molecule 14 and cell entry molecule 14A can be coupled to the intracellular targeting molecule 12. The cell targeting molecule can target a predetermined cell. The cell entry molecule 14A can provide access through the cell membrane to an intracellular region of the cell. Advantageously, the entry does not activate natural immune systems of the cell.

In operation the nanosystem unfolds layer-by-layer to expose the appropriate attached or embedded molecules to accomplish the next step in the sequence for a “nanomedicine” system, as shown in FIG. 1B. To construct the nanodelivery system, the basic nanoparticle is built in process 20. If the nanoparticle exhibits cytotoxicity to the cell, a biocompatible coating can be applied in process 22. A drug or gene delivery molecule can be coupled to the nanoparticle in process 24. A molecular biosensor can be coupled to the drug or gene delivery molecule in process 26. One or more cell targeting, cell entry, and intracellular targeting molecules can be coupled to the system in process 28.

After the nanodelivery system is allowed to enter the a living biological system, validation of the intracellular targeting of the nanodelivery system and activation of molecular biosensor can be performed by use of multi-spectral confocal microscopy in process 30.

Particles

Underlying details of nanomaterials science are known to those of ordinary skill in the art and are believed to be unnecessary to repeat here. But briefly, multilayered nanoparticle systems are usually, but not always, built on a nanoparticle core. These nanoparticles are self-assembled atom-by-atom or molecular layer-by-layer (LBL).

There are two general categories of nanoparticles (with many variations) currently being used for applications in in-vitro and in-vivo nanomedicine: (1) nanoparticle cores with single or multilayered coatings, (2) hollow nanoparticle capsules without cores.

Nanoparticle core materials vary greatly, the most common being made from gold, silica, iron or other magnetic material, or other material. Some are made from magnetic materials which can be useful for recovery of gene products within cells. Such magnetic nanoparticles also act as contrast agents for in-vivo imaging. Some of these magnetic nanoparticles are now commercially available (for example from Miltenuyi Biotec, Germany) and can be used as base core nanoparticles onto which multiple molecular layers can be built to provide a multilayered nanomedical system, according the present disclosure. Most of these nanoparticles must be coated for two general purposes. First, some of them are not water-soluble. To exist and to function in an in-vivo aqueous environment, some of these hydrophobic materials must be marked with a layer of hydrophilic molecules. Second, some of these materials are cytotoxic to cells and tissues. Typically these are covered with lipophilic or other organic molecules to provide a barrier between the cell and the core nanoparticle materials.

Hollow nanocapsules without cores come in a variety of sizes and materials. The simplest ones consist of single or multiple layered liposomes, which are designed to fuse with the lipophilic molecules of a cell membrane and then to spill the contents of the liposome into the interior of the cell. But liposomes, while valuable, have some basic limitations. They are basically one-step systems. They also tend to fuse with the cell surface membrane and, if not protected, the contents of the liposome can be degraded within intracellular vesicles. More complex, layer-by-layer assembly nanocapsules are being made by some research groups (e.g., Lvov and Caruso, 2001) These nanocapsules are self-assembling by alternating charged layers of polymers and similar materials. These nanocapsules are potentially biodegradable and may be less cytotoxic to biological systems, although more detailed studies need to be conducted.

In this disclosure, additional layers can be added to the core, such layers containing drugs or genes to deliver, molecular biosensors, and/or targeting molecules (including both extracellular and intracellular). At least two types of nanoparticles can be used—nanocrystals and nanocapsules. Nanocrystals, self-assembled atom-by-atom and made of semiconductor materials, such as CdTe, available from such sources as the laboratory of collaborator Dr. Nicholas Kotov of University of Michigan, Ann Arbor, Mich. Such nanocrystals can be used to provide core nanoparticles very small (7-10 nm diameter) on which multilayered nanoparticle delivery systems can be constructed. More recently a type of these nanocrystals has become commercially available (“Quantum Dots™” through QDot Corporation, Hayward, Calif.). They also have advantages in terms of brightness of fluorescence and absence of photobleaching during confocal microscopic analyses. Nanocapsules, typically self-assembled layer-by-layer using alternately charged polymers from the laboratory of collaborator Dr. Lvov, Louisiana Tech University, Ruston, La. are typically much larger—on the order of 100 mm in diameter. These nanocapsules can be with or without solid cores and have a larger capacity for holding drugs or genes in their interior. The polymers can also be made from biodegradable polymers, some of which already have FDA approval for in-vivo human use.

Layer-by-layer nanoparticles are formed around a core particle (Lvov, Antipov et al. 2001; Lvov and Caruso 2001). The layers are held together by the charge of the individual molecules, thus they are composed of alternating positive and negative charged species (Lvov, Antipov et al. 2001; Lvov and Caruso 2001). One benefit of using this type of particle is that once constructed, the particle core can be suspended and then made porous by changing solvents without damaging the layers. Incubating these porous nanocapsules with dissolved chemicals, one can load the nanocapsules through diffusion. Once loaded, the nanocapsules can be made non-porous by changing the solvent, thereby encapsulating the chemical of choice. Through this technique one can encapsulate fluorescent dyes and possibly other molecules (Lvov, Antipov et al. 2001; Lvov and Caruso 2001). Reporter genes may also be used as an interior layer because of the inherently negative charge of DNA. Additional layers of targeting molecules may then be added to help direct the particle to the correct cell types (Lvov, Antipov et al. 2001; Lvov and Caruso 2001).

Cells are widely known to only allow particles within a particular size range, 30-200 nm, to pass the outer membrane (Zauner, Farrow et al. 2001). The nuclear membrane is even more tightly guarded, only allowing specific molecules to pass into the nuclear compartment (Stewart, Baker et al. 2001). Passing through these barriers is paramount for the success of nanomedicine. The size of the particle delivered is therefore critical for the success of nanoparticle mediated gene delivery. The major size determinant for nanoparticles is the nature of the core particle, onto which the coats are layered. A variety of specific amino acid localization sequences have been used related to this disclosure to deliver and anchor delivery of molecules to at least three intracellular regions of the cell: (i) the endoplasmic reticulum (ER), (ii) the mitochondria, and/or (iii) the nucleus. Other intracellular components and regions are available for interaction therewith.

Nanocrystals offer several unique advantages over traditional nanoparticles and lipid mediated gene delivery tools. The nanocrystals have spectral properties that are especially convenient for in vitro use (Chan, Maxwell et al. 2002; Dubertret, Skourides et al. 2002; Jaiswal, Mattoussi et al. 2003). These particles fluoresce very brightly and do not photobleach like traditional dyes or fluorescent proteins. These nanoparticles are also small enough (<6 nm) to enter the cell without endocytotic signals. Additionally, a multitude of biological moieties can be bioconjugated to the surface of these tiny crystals. Even with large, biologically active molecules conjugated to the surface of these particles, they remain under 20 nm in diameter.

FIGS. 1C-1F are representations of intracellular targeted delivery. FIG. 1C is a representation of a cell with an uncoated nanoparticle. FIG. 1D is a representation of a cell with a nanoparticle coated with HIV tat fragment. FIG. 1E is a representation of a cell with a nanoparticle coated with anti-CD95. FIG. 1F is a representation of a cell with a nanoparticle coated with a 6× Arginine peptide. Nanocrystals are actually smaller than many proteins, including Streptavidin. Streptavidin labeled nanocrystals were targeted to cells labeled with biotinylated anti-CD95. Live cells were incubated and imaged by multispectral confocal microscopy. Hoechst 33342, an AT base pair specific dye that enters live cells, was added as a counterstain to delineate the nuclear boundary. The purpose of these experiments was to explore the entry mechanisms of semiconductor nanocrystal nanoparticles. Some materials used for nanocrystals, such as CdTe can be cytotoxic without a suitable biologically compatible coating. Further, uncoated CdTe nanocrystals can fail to bind to, or be phagocytosed, by cells. “Naked” nanoparticles (with no biocoatings) did enter some cells non-specifically, showing that biocoatings were not only necessary to target nanoparticles to the cells of interest but also necessary to prevent non-specific uptake. Careful coating and choice of core materials can avoid or lessen concerns of toxicity of the nanocrystals. Interestingly, Streptavidin coated nanocrystals when bound to the surface of cells labeled with biotinylated anti-CD 95 monoclonal antibodies, not only targeted those cells in a highly efficient manner, but also tended to internalize and track to the nuclei of those cells. HIV tat sequences proved highly efficient cell entry and nuclear localization molecules. In at least some experiments, a 6 amino acid peptide of Arginine had a similar effect, although arginine rich amino acid repeat peptides did not prove useful for nuclear localization.

Molecular Biosensors

An important feature of the nanomedical system is molecular biosensors. In at least one embodiment, the biosensors provide molecular error-checking of initial molecular targeting (e.g. antibodies directed against cell surface molecules) based on desired or permissible Boolean logic conditions (based on presence or absence of specific molecules on or within the cell) to reduce false positive targeting which then lowers undesired, adverse bystander side reactions.

Molecular biosensors can also confirm the correct targeting by sensing the intracellular environment and to provide an initial control of drug/gene delivery. In addition, when the drug/gene treatment has resulted in the elimination of the pathogen the absence of the indicator molecules will, and then shut off expression of the introduced gene when the desired response inside a single cell is reached. These secondary checks for correct targeted and control of drug/gene delivery can be used to help minimize “bystander” effects now common as “side effects” in conventional drug administrations to illustrate the use of nanoparticles and molecular biosensors in molecular morphology.

Nanoparticles for in-vivo drug/gene delivery are more complicated, since it is difficult, but not impossible, to construct a similar Boolean targeting scheme on a single nanoparticle. A simpler way to accomplish the objectives is to have a drug delivery system which is binary, i.e., it requires that two different nanoparticles with different targeting molecules bind to the cell (e.g. nanoparticle type 1 with targeting molecule A, and nanoparticle type 2 with targeting molecule B). The intermediate steps bring two different components of the drug/gene delivery system together to form a process that can only be completed if both drug/gene prerequisite molecules are present. The non-specific targeting molecule C can have a nanoparticle with a “suppressor” molecule that blocks the completion of the assembled drug/gene delivery system.

The importance of Boolean combinations of targeting molecules to correctly identify rare cells (the usual in-vivo targeting situation) has been documented, including in previous work by one of the inventors (Leary, 1994). For the reasons given in this reference, the preferred Boolean combination is to have two “positive selectors”, for example A and B, which must be simultaneously present (Boolean AND) and one “negative selector”, for example C, which must be absent (Boolean NOT). This Boolean logic condition of “(A AND B) AND (NOT C)” has been demonstrated in other fields to provide good target selection down to frequencies of more than 10⁻⁶ in at least some applications where each targeting molecule, A or B may only be individually accurate to frequencies of 10⁻². The present disclosure uses a similar Boolean logic sequence to apply to targeting of nanoparticles. The non-specifically binding C target molecule, which should not bind to the correct cells, is used to eliminate cells which may also bind A or B. Targeting molecule C can also be used to eliminate all dead/damaged and other cells which non-specifically bind molecule C. As discussed in the reference (Leary, 1994), A, B and C can also consist of cocktails of targeting molecules. Thus a cell of interest may be identified at A+B+C−. Nanoparticles of different colors can obviously be similarly used for in-vitro, ex-vivo, and in-vivo diagnostic purposes.

In at least one embodiment, a triple-fusion protein molecular biosensor can be designed and constructed that locates its target cell or cellular compartment in a subcellular domain localization sequence, detects the presence of the target molecule based on the specific reaction between it and the biosensor, and releases a signaling molecule that in turn generates a reporter molecule. Stated differently, a biosensor molecule can have at least three domains: first, a homing sequence that will guide the protein to the compartment in the cell where detection needs to occur; second, the detector or cleavage domain that is activated upon contact with the intracellular target sequences (an enzyme), i.e. a peptide sequence that is recognized by a specific protease; and third, an activator element that will induce transcription of a target gene upon cleavage of the detector sequence. The activator element can be a signaling molecule that is released to interact with the nucleus to produce a gene product under the action of promoter sequences.

The present disclosure also provides for manufacturing genes and/or chemicals within the living cell. The central idea is to set up gene therapy “nanofactories” inside single living cells. The therapeutic gene can be “manufactured” in-situ (i.e. inside the cell) using these gene templates and local ingredients already within a cell molecular biosensors linked to these genes control their expression. The nanofactory can include a combination of one or more biosensors, promoter sequences, and therapeutic genes linked together to operate much like an (artificial) virus does when it uses host cell raw materials and machinery to manufacture its viral proteins, RNA, or DNA.

If these biosensor molecules are “upstream” of the promoter sequences and the therapeutic gene, the biosensor can be used to turn on (or off) the production of copies of the therapeutic gene. Gene delivery is started in response to a biosensor detected problem; gene delivery is halted when the cell response indicates that more gene therapy is not needed.

Nanomaterials are inherently self-assembled atom-by-atom, or layer-by-layer (LBL). A way to do this is to use alternately charged polymer layers that also contain antibodies, or other targeting molecules such as aptamers, intracellular localization amino acid sequences, molecular biosensors, and therapeutic genes. The first layer on the core would represent the last step on the programmed sequence of nanomedical events. Each of these charged layers contributes to the overall “zeta potential” (an oversimplification but essentially the “net” charge of the nanoparticle as seen at a distance) of the nanoparticle which must interact with a charged living cell. A living cell typically has a fairly highly negatively charged cell surface layer of molecules. If the nanoparticle were positively charged it would stick non-specifically to cells and destroy any specificity of targeting. The zeta potential of both nanoparticles and cells changes according to environmental factors such as pH and ionic strength of the surrounding fluid. Since the composition of the cells is what it is, the nanoparticle zeta potential, and the zeta potential of the remaining nanoparticle portion as each layer is stripped away during the multi-step process will have the desired charge structure when they are in the particular pH and ionic environment inside the cell to prevent non-specific interactions, such as by adjusting the charge structure.

Having described the general principles of the present disclosure, attention is turned to specific experiments and exemplary embodiments using the principles disclosed herein. It is expressly stated and to be understood that the experiments are exemplary and non-limiting as other embodiments are application and contemplated given the principles described herein.

Experiment 1

One exemplary biological model was chosen as for illustration regarding Hepatitis C virus (HCV) infection of single cells. The biosensor is targeted to the same sub-cellular location where the HCV proteins are synthesized. Only when the viral protease is detected, would the expression of an anti-HCV gene product be triggered.

The nanoparticle can be built on a nanoparticle core of polystyrene, silica, gold or other material. A multicomponent HCV biosensor was constructed. This protease activated biosensor is a triple fusion protein consisting of a transactivator, cleavage, and localization domains that should target the protein to the perinuclear region. The transactivator region functions to activate transcription when released from the localized biosensor that is anchored to the targeted endoplasmic reticulum (ER). This anchored protein cannot move to the nucleus and initiate transcription due to the cytoplasmic localization, thus the transactivator is restrained such that it cannot move to the nucleus and bind to DNA. The cleavage domain separates the transactivator from the anchor region and is designed to the enzymatic activity of a specific protease. If the appropriate protease is present and cleaves the substrate, the transactivator released from the anchor domain end is then free to induce transcription within the nucleus.

FIG. 2 is a schematic illustrating a spectral unmixing algorithm implemented on a multispectral confocal microscope, analyzing the activity and results. An important technology to visualize and study the proper localization of nanoparticles to their intracellular targets is confocal microscopy. By knowing directly or indirectly, all of the spectral contributions of each dye or probe plus the autofluorescence spectrum of a cell, the colors can be “unmixed” on a pixel-by-pixel basis on each plane of a multi-plane confocal image. The algorithm essentially fits the overall color curve using regions of each dye or component spectrum that are less contaminated with the overlap of other colors. The resulting emission fingerprinting technique can be superior to the use of conventional optical filters which still leave considerable optical overlap.

The method of spectral deconvolution was developed at JPL/Cal Tech (Pasadena, Calif.) and was implemented in a new generation of multispectral confocal microscope (Model 510 META, Zeiss, Inc.). The basics of the method are shown in FIG. 2. The “emission fingerprinting” algorithm (Bearman et al. 2002) works by fitting the spectral components over low or non-overlapping portions of the combined spectrum of a multicolor image. The components are appropriately weighted so that the combination of color components matches the overall spectrum from the image pixel-by-pixel in each image plane.

Multi-spectral confocal microscopy was used as a validation tool for both nanoparticle/biosensor targeting and intracellular localization. Since multiple fluorescent colors were used to label biosensors, cell membranes, endoplasmic reticulum and nuclei, it became necessary to use a multispectral confocal microscope that could separate the color optical overlaps.

Results and Discussion:

Nanoparticle Targeting

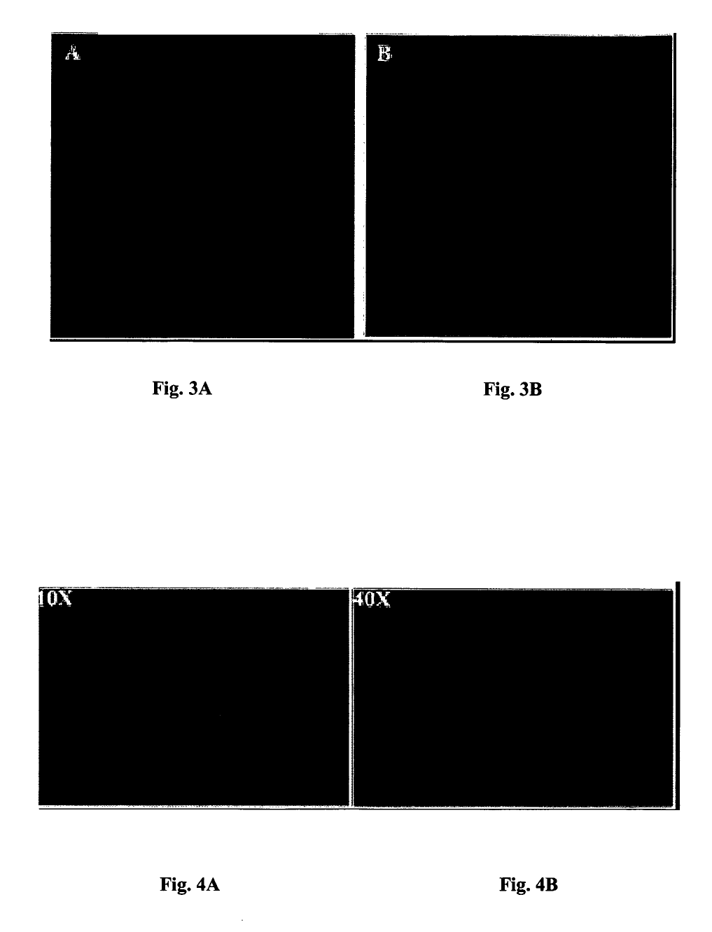

FIG. 3A is a representation of a photomicrograph of a conventional antibody labeling system. FIG. 3B is a representation of a photomicrograph of a nanoparticle antibody labeling system and will be described in conjunction with FIG. 3A. Initial experiments were conducted to compare the behavior of conventional antibody staining and nanoparticle labeling. In FIG. 3A, live human BJAB cells were directly-labeled with FITC-conjugated anti-CD95 IgG, whereas in FIG. 3B, live BJAB cells were indirectly-labeled with unconjugated anti-CD95 IgG, and then incubated with a secondary reagent constructed of FITC-conjugated 500 nm polystyrene nanoparticles coated with a secondary goat anti-mouse antibody against IgG. The nanoparticle labeling system showed similar results to that of the conventional antibody labeling system. These very large 500 nm nanoparticles were used in order to permit direct optical visualization by fluorescence microscopy.

FIG. 4A shows a 10× objective phase-fluorescence photomicrograph of the combined mixture of cells and nanoparticles. FIG. 4B shows a 40× two color fluorescence only photomicrograph and will be described in conjunction with FIG. 4A. To test the targeting accuracy and efficiency of the nanoparticle system, non-targeted, CD95-negative, MOLT-4 cells were labeled with CellTracker™ Blue CMAC (7-amino-4-chloromethylcoumarin), a fluorescence tracking dye (Molecular Probes, Eugene, Oreg.). Green fluorescent nanoparticles were added to a mixture of CD95 positive BJAB cells, previously labeled with unconjugated anti-CD95 antibody (non-fluorescent), and MOLT-4 cells, previously labeled with CMAC, was made. Green fluorescent nanoparticles (as described in FIG. 3) were then added to the cell mixture. While not all of the CMAC-negative BJAB cells were labeled at this ratio of nanoparticles and cells (FIG. 4A), none of the CMAC-positive, CD95-negative MOLT-4 cells bound nanoparticles (FIG. 4B). The MOLT4 cells were used as a negative control because they do not normally express CD95 on their surface whereas BJAB cells were used as a positive control because most, but not all, BJAB cells constitutively express CD95 on their surface. Only the BJAB (CMAC negative) cells, that are CD95 positive, were bound to the green nanoparticles.

Biocompatibility of Nanoparticles

While molecular membrane transport facilitating sequences were being developed, the biocompatibility of CdTe semiconductor material nanocrystals to living T24 human cells was tested by direct microinjection of nanocrystals into living cells using a Narashige microinjection system mounted on to an inverted phase-fluorescence microscope (Nikon DiaPhot). Initial results showed considerable cytotoxicity in the absence of biocoatings being applied to the nanoparticle surface. Coating the nanocrystals with either galactosamine or Lipofectamine 2000 (Invitrogen, Inc., Carlsbad, Calif.) was found to reduce this cytotoxicity to control levels for the time span of these experiments. Lipofectamine also provides cell entry molecules.

Molecular Biosensor Design

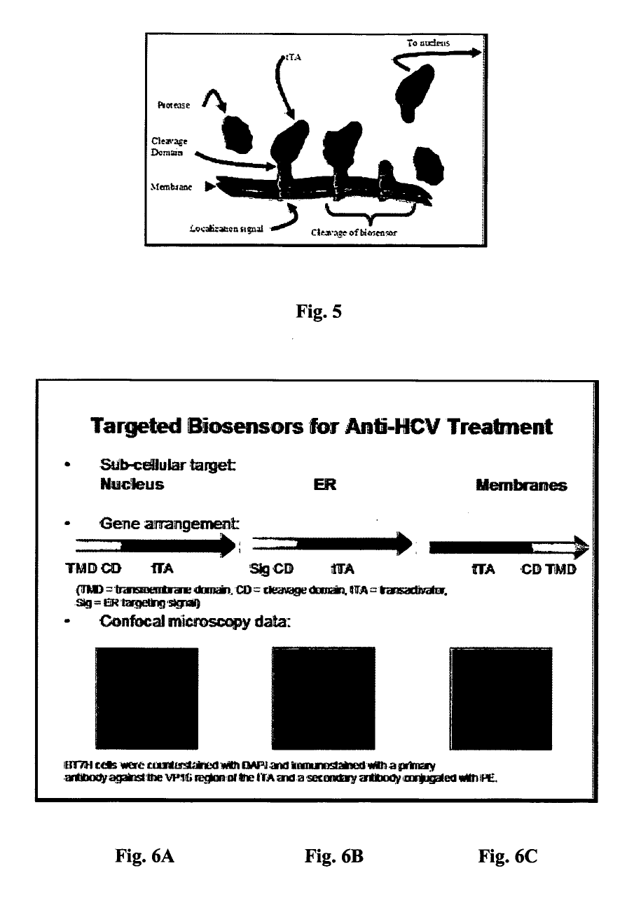

FIG. 5 is a schematic diagram and sequence of one embodiment of a molecular biosensor. In at least one embodiment, the disclosure provides a design and construction of a triple-fusion protein molecular biosensor that locates its target cell or cellular compartment, detects the presence of the target molecule based on the specific reaction between it and the biosensor, and release of a signaling molecule that in turn generates a reporter molecule. The protease based biosensor can contain a tetracycline inactivated transactivator (tTA), a protease specific cleavage domain, and a localization signal. The activated protease cleaves the cleavage domain, releasing the tTA. The tTA then localizes to the nucleus and activates transcription of a predetermined gene. The overall biosensor construct must contain a subcellular domain localization sequence, a cleavable segment that is activated upon contact with the intracellular target sequences (an enzyme), and a signaling molecule which is then released to interact with the nucleus to produce a gene product under the action of promoter sequences.

The data obtained by use of the construct shown in FIG. 5 is shown in FIGS. 6A, 6B, 6C. Measurements were made of the intracellular localization of molecular biosensors BS-1, BS-2, and BS-3 (three different sequences) targeted molecular biosensors in BT7H cells. Confocal images from BT7H cells transfected with molecular biosensors BS-1 (FIG. 6A), BS-2 (FIG. 6B), or BS-3 (FIG. 6C) were stained for biosensor (red) and the DNA counterstained with DAPI (blue). The results show successful targeting to the nucleus, to the endoplasmic reticulum, and to the plasma membrane.

Intracellular Localization of Molecular Biosensors

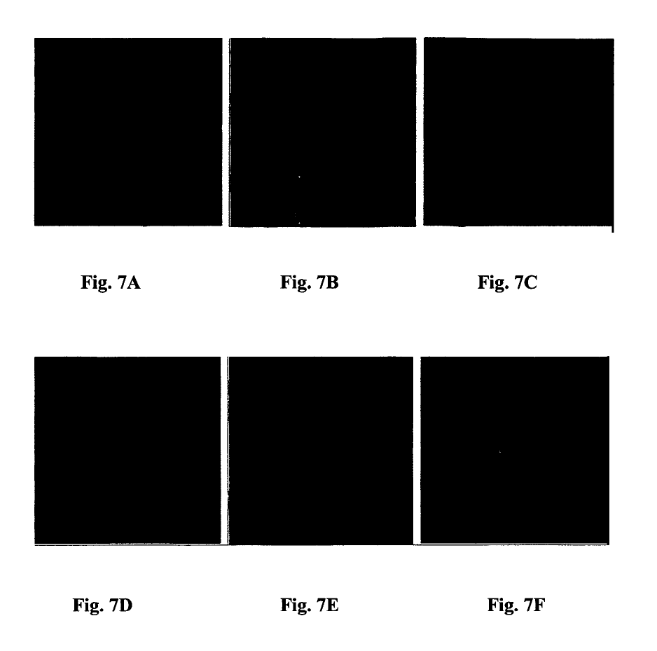

FIGS. 7A, 7B, 7C, 7D, 7E, 7F represent photomicrographs of intracellular co-localization of molecular biosensors and will be described in conjunction with each other. More specifically, the photomicrographs represent three color multi-spectral confocal intracellular co-localization of molecular biosensors and NS3-specific flavivirus protein in Huh7 cells data obtained from the construct shown in FIG. 5. As discussed above, a biosensor molecule can have at least three domains: (i) a homing sequence that will guide the protein to the compartment in the cell where detection needs to occur; (ii) the detector or cleavage domain, i.e. a peptide sequence that is recognized by a specific protease; and (iii) an activator element that will induce transcription of a target gene upon cleavage of the detector sequence. Protease containing Huh7 cells were transfected with nothing (FIG. 7A), or molecular biosensors BS-2 (FIGS. 7B, 7E), BS-3 (FIG. 7C, 7F), and pTet-Off (FIG. 7D). FIGS. 7B, 7C show what appears be inactivated biosensor (BS) proteins that seem to localize within the cytoplasm. Panels 8D, 8E, and 8F show cells with tTA, BS-2, or BS-3 throughout the entire cell, which indicates activated BS proteins. The cells shown in FIG. 7D were transfected with only the transactivator portion of the BS protein and therefore serve as a positive control for the activated BS protein from either BS construct. FIGS. 7D, 7E, 7F were found to have BS throughout the z axis, as opposed to Panels 8B and 8C that were found to have large regions without protease or BS proteins. Because previous experiments described the localization of the BS proteins to the nucleus, all cells were counterstained with a fluorescent phallotoxin (actin stain, blue) to visualize the localization of the BS proteins (green) with respect to the entire cell. The NS3 protease was also immunostained and is shown in red.

Experiment 2

Another exemplary biological model is for illustration is cellular radiation damage to single cells as is likely to occur during long term/deep space missions by astronauts. The principles of the present disclosure can result in a gene therapy technique that would provide increased in vivo protection against radiation damage to the blood and bone marrow of astronauts who experience long term/deep space missions. The disclosure thus provides a method and process of an in vivo intra-cellular DNA repair system for astronauts to repair radiation damaged cells that had suffered radiation damage before they progress to radiation induced leukemia or other diseases.

In general, the disclosure provides for seeking out radiation-damaged cells by providing targeting nanoparticles. A nanoparticle enters a cell and delivers a gene to detect an expression of a biosensor. If there is an expression, a determination is made as to whether the damage is repairable. If the damage is repairable, then a therapeutic DNA repair gene is expressed. If the damage is irreparable, then the cell can be programmed to induce apoptosis to lessen the risk of inflammatory responses of the immune system which can, in many diseases and injuries, be of greater danger to the person than the disease or injury itself. For example, it can be beneficial to not allow cancerous cells to arise from mutations produced by radiation damage. The biosensor can continue to sense for a time, whether damage is present. To test the feasibility of these approaches, an antioxidants-sensitive biosensor (Zhu and Fahl, 2000) was attached to DNA repair enzymes, MutY/Fpg, previously designed and prepared in the outside laboratory.

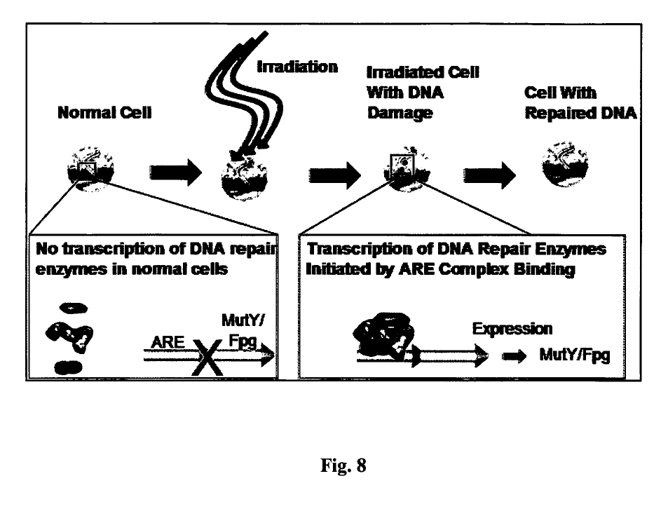

FIG. 8 is a schematic diagram of the transcription of the DNA repair enzymes initiated by ARE complex binding. When a cell undergoes oxidative stress, it produces a series of molecules which will bind to the anti-oxidant response element (ARE). The binding of these molecules can be used as a molecular biosensor to drive the synthesis of the above special DNA repair enzymes.

This ARE biosensor, attached to a green fluorescent protein reporter sequences (eGFP), was tested in cultures of T24 cells subjected to tert-butylhydroquinone (tBHQ), a chemical which produces oxidative stress in cells in a manner similar to UV radiation, but which is more easily dosed and controlled. The cell detects ROS and activates transcription factors which bind to specific promoter regions. Transcription downstream of these response elements is then activated. The construct used was constructed by Zhu and Fahl, 2000. This construct is composed of a number of antioxidant response element (ARE) repeats followed by a minimal thymidine kinase promoter, ahead of a EGFP reporter gene. The sensitivity of the biosensor is dramatically affected by the number of ARE repeats, with four repeats giving optimal sensitivity (Zhu and Fahl, 2000). The activity of this biosensor can be seen in T24 cells through the addition of 100 μM tert-butylhydroquinone, an inducer of ROS (Sigma-Aldrich Chemical, Inc., St. Louis, Mo.). T24 cells are a human cell line derived from a transitional cell carcinoma that constitutively expresses CD95 on the surface (Mizutani et al. 1997) (American Type Culture Collection, Manassas, Va.). Cells showing signs of oxidative stress were detected as green fluorescent positive cells as shown in FIG. 8.

In at least one embodiment of nanomedicine for astronauts, up-regulation and transport of the CD95 molecule to the radiation (or oxidative stress) damaged cell can be used. Amounts of cell surface CD95 vary in roughly a dose dependent manner with radiation exposure (Sheard, 2001). So, CD95 serves as the initial surrogate biomarker for radiation damage. Radiation dosed cells with two cell lines have been modeled with the present disclosure, one of which expresses no CD95 (human MOLT-4 monocyte cell line) and another cell line (BJAB) which expresses high quantities of cell surface CD95. Once inside, the nanoparticle system performs a secondary check for oxidative stress which is highly correlated to radiation exposure using a biosensor sensitive to the presence of reactive oxygen species molecules. Since exact radiation exposure is difficult to control, in initial studies have used a chemical which produces the same oxidative stress as radiation but in an easily dosed manner.

This system would therefore use a promoter based sensor which could detect the presence of reactive oxygen species (ROS). When ROS is present, the sensor triggers the transcription of foreign DNA repair enzymes, which can be an alternate to the human innate DNA repair system in repairing the increased level of radiation induced DNA damage and cellular radiation damage to single cells as is likely to occur during long term/deep space missions by astronauts. Further, the system can include a reporter gene, such as eGFP reporter gene, coupled to the biosensor that fluoresces a color, such as green or red, when activated.

The nanoparticle can be built on a nanoparticle core of polystyrene, silica, gold or other material. For detection of reactive oxygen species in chemically or radiation-damaged cells, an ROS molecular biosensor constructed based on previously reported sequences (Zhu and Fahl, 2000) was used. Other biosensors are certainly possible, in keeping with the underlying concepts of the present disclosure. The ROS biosensor is being used to identify and help induce the expression of DNA repair enzymes during times of increased oxidative stress. The antioxidant response element was coupled to an EGFP (enhanced green fluorescent protein) reporter. Zhu and Fahl (2000) first adapted this promoter for use as an in vitro assay for cells experiencing oxidative stress. The most sensitive of the constructs contained four repeats of the antioxidant response element followed by the EGFP coding sequence of the gene and a poly A tail as explained further in Arizona Cancer Center, (Zhu and Fahl 2000).

In this second application area, involving the detection of oxidative stress and DNA damage caused by either chemical agents or radiation, a reactive oxygen species (ROS) biosensor was used. This biosensor can be, for example, a promoter based biosensor (Fahl and Zhu, 2000), composed of three elements: a series of response elements (EpRE), minimal thymidine kinase promoter (TK), and a reporter gene (green fluorescent protein (GFP)).

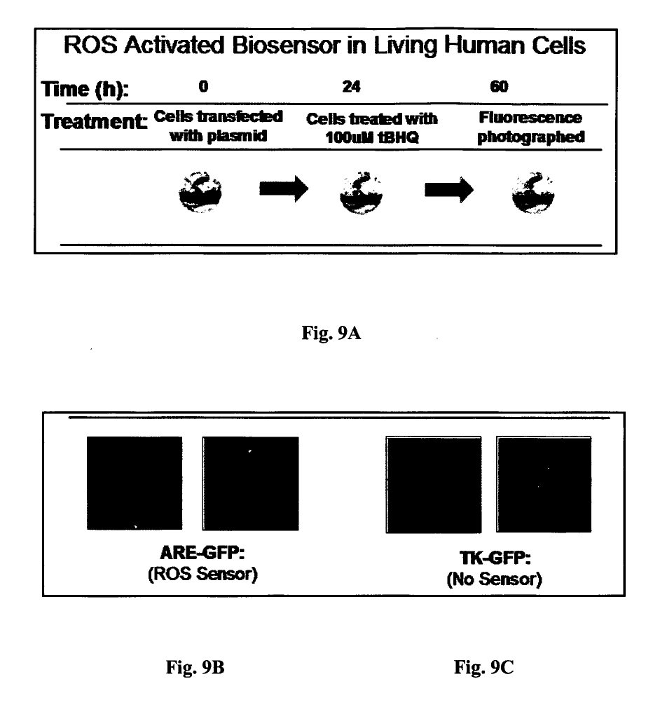

FIG. 9A is a schematic representing a progression of ROS treated cells. FIGS. 9B, 9C represent photomicrographs of the cells with an ROS sensor and without a sensor, respectively. The figures will be described in conjunction with each other. Cells were transiently transfected at ˜60% confluence with either ARE-GFP or TK-GFP and at 24 hours post transfection the cells were treated with tert-butylhydroquinone (tBHQ), an ROS inducing agent. The cells were examined every 12 hours post treatment. Weak GFP fluorescence was present at 48 hours after treatment, and stronger GFP fluorescence was observed after 60 hours and photographs were taken. A fraction of cells, expected at this concentration of tBHQ showed signs of oxidative stress.

Experiment 3

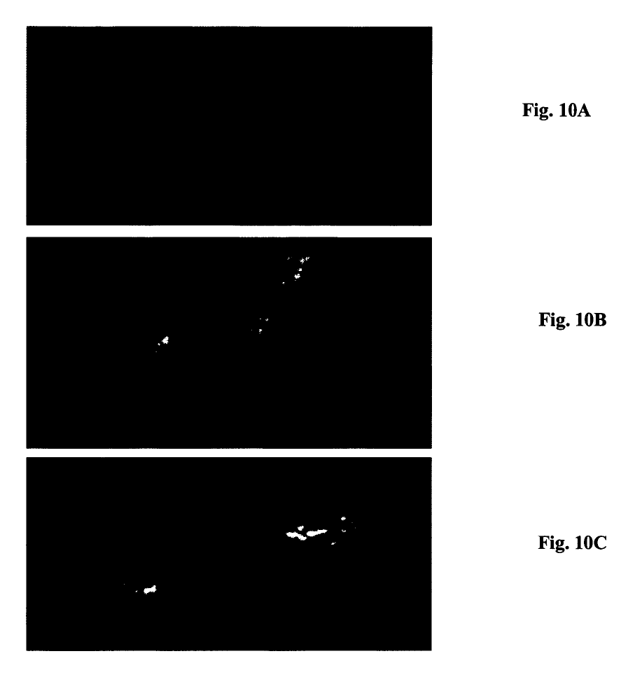

FIG. 10A is representation of a photomicrograph of results of a DNA repair enzyme with no localization anchoring sequence. FIG. 10B is a representation of a photomicrograph of results of a DNA repair enzyme with a mitochondrial localization anchoring sequence with transient expression. FIG. 10B is a representation of a photomicrograph of results of a DNA repair enzyme with a mitochondrial localization anchoring sequence, with stable expression. The figures will be described in conjunction with each other.

Some living organisms have a second repair pathway, not normally expressed in humans. One enzyme, glycosylase, is absent in normal human cells. This experiment tested whether nanomedicine according to the teachings of the present invention could be used to activate a second repair pathway. Preliminary test results indicated that such a mechanism could be used, and reduced the repair time of more than 24 hours using the usual DNA repair pathway functioning in normal human cells down to 6 hours by the assisted DNA repair transient gene therapy approach.

This normally absent repair mechanism was activated by transfection of a glycosylase containing gene sequence which also contained an eGFP reporter molecule. UV damage can occur in both nuclear and mitochondrial DNA. Since many molecules introduced into a cell frequently track non-specifically to the nucleus, the ability of intracellular localization sequences to guide these repair molecules to the mitochondria could enhance the repair.

The T4 transfected DNA repair enzyme with no localization anchoring sequence creates a diffuse fluorescence, as shown by the diffuse staining in FIG. 10A. The repair molecules did not appear to track to any specific region of the cell. The nanoparticle included a DNA repair enzyme construct Wt-T4-PDG-GFP in CHO-XPG, where Wt (“wild type”) with no localization sequences, the T4 is the standard bacterial promoter T4, PDG is part of the DNA repair enzyme, GFP is an enhanced green fluorescence protein reporter molecule, and CHO is Chinese Hamster Ovary, clone XPG. The CHO-XPG cell line can be transfected transiently, but not permanently, with the repair enzyme—an example of transient gene therapy, where the gene is no longer expressed after a few days. The DNA repair enzyme, when not hooked up to a localization sequence, diffused itself in the interior of the cell to the point where it lacked enough concentration to be useful in terms of DNA repair.

In contrast, the T4 transfected DNA repair enzyme with a mitochondrial localization anchoring sequence resulted in positive tracking to the mitochondria with a transient expression. The nanoparticle included a DNA repair enzyme MLS35-T4-PDG-GFP in CHO XPG, where the MLS35 localization sequence helped guide the rest of the DNA repair enzyme and reporter molecule to the mitochondrial DNA where it was able to successfully repair that DNA because it was now in sufficient concentration to be effective. Similarly, the T4 transfected DNA repair enzyme with a mitochondrial localization anchoring sequence resulted in a positive tracking to the mitochondria with a stable expression. The nanoparticle included a DNA repair enzyme MLS18-T4-PDG-GFP in hXPA, where the repair enzyme transfected permanently into a different type of cell line that allowed it to be stably expressed—an example of permanent gene therapy which does not turn off forever, but merely turns on and off as needed. Transient gene therapy serves as just another form of a manufacturable “drug” without all of the concerns of permanent gene therapy.

To test the actual effectiveness of these DNA repair enzymes inside living cells, comet assays were performed. The “comet” assay (Tice, 2000) shows the attempt of cell trying to repair its DNA strand breaks as a comet-like tail. When successful in DNA repair, the comet tail is eliminated. Initial stage test results showed that repairable human cells can be repaired in six hours by this assisted DNA repair transient gene therapy approach, as compared with a required repair time of more than 24 hours using the usual DNA repair pathway functioning in normal human cells.

Experiment 4

As discussed earlier, one of the issues limiting nanoparticle implementation into nanomedicine is the cost and complexity of constructing nanoparticles. In at least one embodiment, the present disclosure provides a magnetic nanoparticle system. Magnetic nanoparticles can not only be used to deliver genes, but also that those same genes can be recovered from cells after expression for several days. The construction of layered nanoparticles can be accomplished in a relatively short time using commercially available biological components. Testing the nanoparticles for DNA bioconjugation has been accomplished in at least one series of experiments, where the nanoparticles were able to deliver genes to a human hepatoma cell line, Huh-7. It was found that tethering orientation was relevant to gene expression. Several days after exposure to nanoparticles, the nanoparticle tethered genes could be recovered and amplified from populations. This data demonstrates the stability and biological usefulness of the DNA tethered to these nanoparticles. Further, DNA tethered magnetic nanoparticles were capable of transfecting tethered genes in vivo.

Because these agents are used primarily in diagnostic in vivo imaging, many of the particle formulations are already approved for use in humans. The magnetic properties of these particles are quite favorable for layered construction of a non-viral based gene delivery vector. One of the most difficult challenges facing researchers constructing layered nanoparticles is the purification of the particles after each step. With magnetic particles, the purification is simplified. This disclosure and related experiments represents a breakthrough in DNA tethered magnetic nanoparticles for in vitro and in vivo gene delivery and subsequent recovery. The magnetic properties of nanoparticles have been used to enhance gene transfer for gene therapy applications (Plank, et al., 2003, Krotz, et al., 2003, Scherer, et al., 2002). In this case the nanoparticles were used to concentrate the plasmid to a specific location and thereby increase the likelihood of transfection (Scherer, et al., 2002). This group used clusters of plasmid DNA and coated magnetic nanoparticles to target cells using the magnetic properties of the nanoparticle clusters (Plank, et al., 2003).

In general, superparamagnetic nanoparticle cores coated with streptavidin were chosen for gene transfer and recovery because they were easily obtainable, relatively simple to construct, and could be purified from the layer components using magnetic columns. The core particles are composed of an iron oxide core coated with dextran and bioconjugated to streptavidin, with the complete particle measuring approximately 50 nm in diameter. The disclosure provides a relatively simple procedure for DNA conjugation, purification, delivery to cells and recovery of these nanoparticles. These particles were found to have reasonably high expression, with respect to free DNA, when coated with Lipofectamine 2000 (Invitrogen, Inc.). It was found that intact nanoparticles could be recovered from populations of positive and negative cells through purification and PCR analysis. These nanoparticles were also able to deliver genes in vivo.

More specially, the materials and methods are as follows:

Biotin Labeled DNA Fragment Preparation

PCR amplification was used to create biotin labeled DNA fragments. Oligonucleotide primers were purchased from Integrated DNA Technologies, Inc. For initial studies, either the 5′ or the 3′ oligo was made with a single biotin tag. The sequences were based on the pEGFP-C1 (BD Clontech, Inc.) template: forward 5′-TAG TTA TTA ATA GTA ATC AAT TAC GGG GTC ATT AG-3′, reverse 5′-TAC ATT GAT GAG TTT GGA CAA ACC ACA ACT AGA AT-3′ (integrated DNA Technologies, Inc.). Later studies used only 5′ oligos labeled with biotin. These oligos were then used as PCR primers. A typical reaction would include 25 ul Red Taq, (Sigma Chemicals, Inc.), 1 ul 5′ biotinylated primer, 1 ul 3′ primer, 1 ul template, to 50 ul with water. The primers were at 200 pM and the template at 50 ng/ul. A typical reaction for DNA tethering to magnetic nanoparticles would include about 25 of these reactions. Typical PCR cycles would include about 35 cycles of denaturing temperature at 94° C. for 30 seconds, annealing temperature at 65° C. for 30 seconds and extension for 2 minutes at 72° C.

DNA Tethered Magnetic Nanoparticle Construction

Biotin labeled PCR products were tethered to streptavidin coated magnetic nanoparticles (Miltyni Biotech, Inc.). DNA tethered magnetic nanoparticles were constructed as per the manufacturer's instructions. Briefly, the magnetic nanoparticles were incubated with the biotin labeled PCR fragments at the ratio prescribed by the manufacturer. The mixture was allowed to sit at room temperature for 30 minutes. During that time, the magnetic column was prepared by washing once with the 100 ul of the included nucleic acid buffer and three times with 100 ul Optimem (Gibco, Inc.). Once washed, the column was loaded with the DNA nanoparticle mixture. The column was then washed three times with 100 ul Optimem. The nanoparticles were eluted by removing the column from the magnet and adding the 100 ul of Optimem. The resulting brownish solution contained DNA tethered nanoparticles.

Lipid Coating of DNA Tethered Magnetic Nanoparticles

The DNA tethered nanoparticles were coated with Lipofectamine 2000 as per the manufacturer's instructions for DNA. The DNA tethered magnetic nanoparticles were treated as DNA for lipid coating. Briefly, the eluted particles were diluted in the appropriate amount of Optimem and incubated for 5 minutes at room temperature. An appropriate amount of Lipofectamine 2000 was diluted in a separate tube and incubated at room temperature for 5 minutes. After 5 minutes, the two tubes were mixed gently and combined. This mixture was allowed to stand for 20 minutes before added to the cell culture.

Confocal Microscopy

Cells were examined with a Zeiss 510 META confocal microscope. Excitation wavelengths included UV and/or 488 nm depending on the fluorescent probes used. Appropriate emission wavelengths were determined and used for each fluorophore used. Cells were analyzed with either 20, 40, 63, or 100× objectives. A 20× objective was used for imaging large numbers of cells for analysis.

Image Analysis

A standard wave-propagation algorithm was used to segment the images over a singular threshold. An upper and lower bound were chosen for sub-segmentation. Segments which fell below the lower area bound were removed. Segments which were above the upper bound were re-segmented with a higher threshold and reexamined. The threshold level was computed as the average of the intensity of the pixels within the segment minus the standard deviation of the intensity of the pixels bounded below by zero. Threshold levels are computed individually for each sub-segment. The output is a list of segments associated with a bitmap representing the segment, the total intensity, area and standard deviation of intensity for that segment (23).

Cell Culture and Transfections.

Cells were incubated at 37 C in 5% CO2. The Huh-7 cell line, derived from a human hepatoma, was cultured in DMEM supplemented with 10% FBS (Sigma, Inc.) and Penicillin/Streptomycin (Sigma, Inc.). Cells were transiently transfected with Lipofectamine 2000 (Invitrogen, Inc.) according to the manufacture's instructions. Each experiment was done at least in triplicate and positive and negative controls were present in all experiments.

Results:

Conjugation of DNA to Magnetic Nanoparticles

The initial construction of the DNA tethered nanoparticles began with the creation of biotin labeled DNA fragments containing the minimal genetic material to be constitutively expressed in mammalian cells. Biotin labeled PCR primers were used to generate CMV-EGFP-pA containing DNA fragments (1.5 kb) with 5′ biotin labeled, 3′ biotin labeled, and unlabeled. These fragments contain all of the information needed to express pEGFP-C1 from within the nucleus and were conjugated to magnetic nanoparticles (magnetic nanoparticles) for transfection.

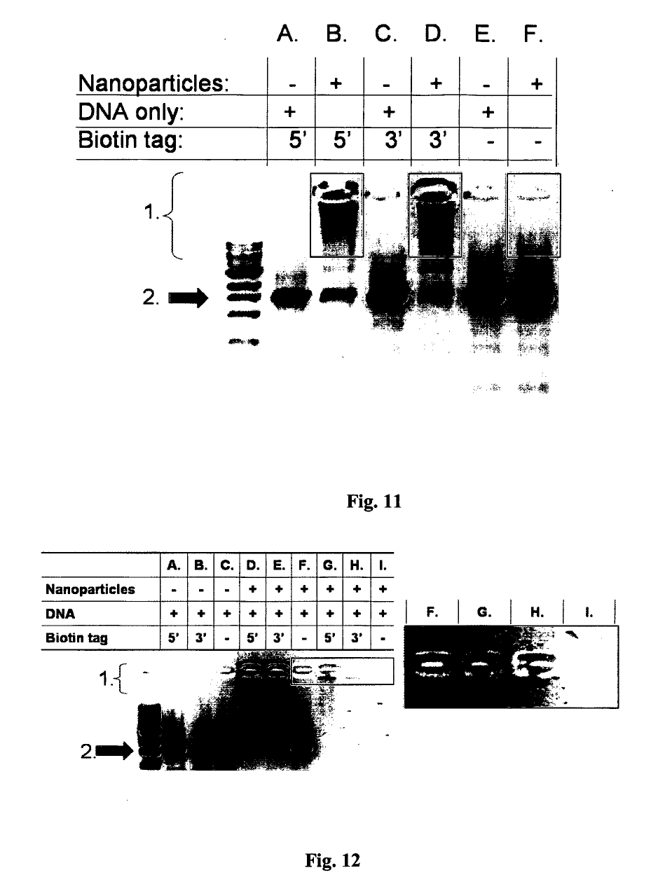

FIG. 11 is a schematic representation of a conjugation of DNA to a magnetic particle (MN). Streptavidin coated magnetic nanoparticles were incubated with each of the DNA fragments and analyzed by agarose gel electrophoresis. Lanes A, C, and E contained only the PCR product. Lanes B, D, and F contained magnetic nanoparticles incubated with the PCR fragments. Only the DNA in Lanes A to D contained biotin tags. DNA in lanes E and F contained no biotin tag and were therefore used as negative controls. The black squares indicate where high molecular weight or uncharged DNA runs. The dark staining seen in Lanes B. and D. indicates that the DNA was able to bind to magnetic nanoparticles and was now trapped at the top of the gel due to its large size. This gel also shows that there is a significant amount of unbound DNA present. Because of this, the magnetic nanoparticles need to be purified from the contaminating free DNA fragments as described in the next section.

Removal of Free DNA from Magnetic Nanoparticle/DNA Solutions

FIG. 12 is a schematic representation of a purification of DNA tethered MN, where the right portion of the Figure represents a magnified image of the box around Lanes F to I. In these experiments, the mixtures of DNA and magnetic nanoparticles were washed four times to remove unbound DNA using a magnetic column. It was found that the magnetic properties of these particles enabled the rapid purification of the magnetic nanoparticles from the DNA solution. These samples were then run on an agarose gel. Lanes A to C represent only DNA fragments. Lanes D to F contain the magnetic nanoparticle/DNA mixture. After washing, a portion of the magnetic nanoparticles were run onto this gel (Lanes G to I). If carefully examined, dark staining can be seen only in lanes G and H near the loading well. This suggests that the free DNA has been removed and only the DNA tethered magnetic nanoparticles remain in solution.

Expression Levels of Cells Transfected with DNA Tethered Magnetic Nanoparticles

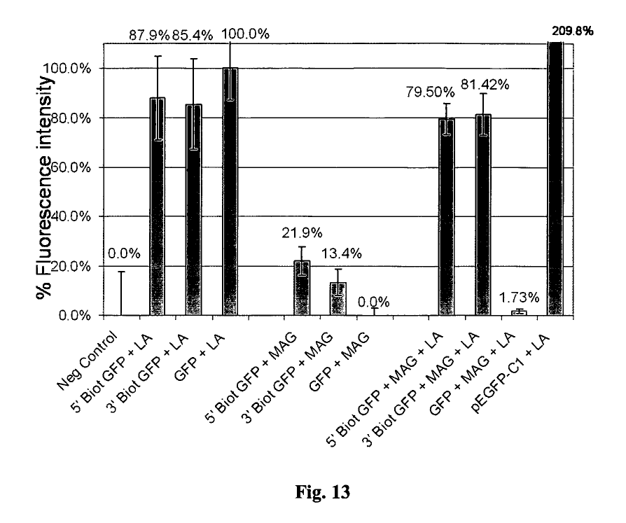

FIG. 13 is a schematic representation of expression levels of EGFP from DNA tethered MN. After washing, the DNA tethered magnetic nanoparticles were mixed with Lipofectamine 2000 in order to enhance transfection into cells (24). This complex was then delivered to cells cultured in chamber slides, incubated for 48 hours, fixed, permeablized, and finally counterstained with DAPI. In vitro gene expression levels are shown as the % of Lipofectamine transfected EGFP DNA (EGFP+LA). Biotin tagged DNA was also transfected into cells (Biot EGFP). Cells transfected with DNA bound nanoparticles are labeled BIOT+MAG. Slides were subsequently photographed under fluorescence using a Nikon CoolPix 990 digital camera. The images obtained were then analyzed with an in house slide based cytometry software program and the resulting data presented in FIG. 13. Note that all of the values were normalized to the samples treated with the un-labeled GFP fragment transfected with Lipofectamine 2000. The intact pEGFP-C1 plasmid was found to be about two times the expression level as the un-labeled GFP fragment. Finally, the unlabeled DNA exposed magnetic nanoparticles as expected did not show any appreciable expression of GFP.

In Vitro Gene Expression with DNA Tethered Magnetic Nanoparticles

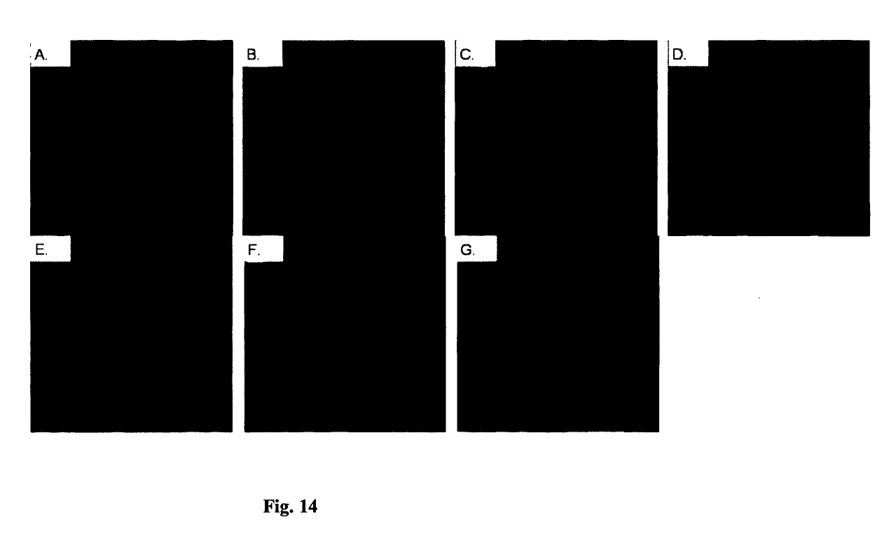

FIG. 14 is a schematic representation of an expression of EGFP from MN tethered to EGFP DNA. Panel A is an image of untreated cells. Panels B, C, and D represent cells transfected with DNA fragments with 5′, 3′, and no biotin tags using Lipofectamine 2000. Panels E, F, and G show cells exposed to DNA tethered to magnetic nanoparticles from 5′, 3′, and no (control for free DNA) biotin tags; these particles were also coated with Lipofectamine 2000. DNA tethered to magnetic nanoparticles showed similar levels of gene expression as those same free gene sequences directly transfected into these cells.

Recovery of DNA Tethered Magnetic Nanoparticles

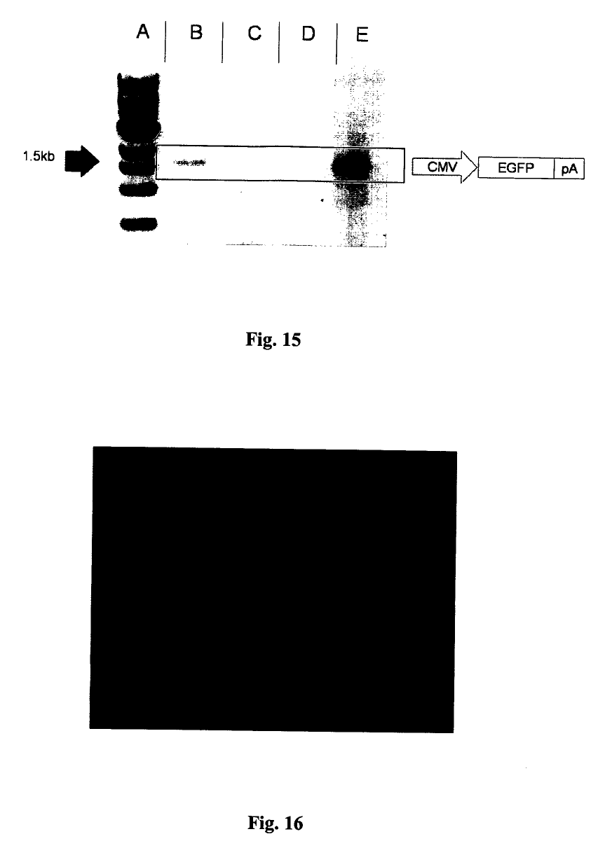

FIG. 15 is a schematic representation of recovery and PCR of DNA tethered to magnetic nanoparticles, where lane A represents a 1 kb marker, lane B represents 5′ tethered, lane C represents 3′ tethered, lane D represents no tether; and lane E represents positive control. These data demonstrate that DNA tethered nanoparticles could be recovered from in Huh-7 cells 72 hours post-treatment. Cells were lysed and the magnetic nanoparticles were purified from the cell lysate with magnetic column separation. The nanoparticles were then eluted from the column and concentrated. The resulting solutions were then used as templates for PCR reactions. The resulting Panels were then run on an agarose gel, showing the PCR products of DNA tethered magnetic nanoparticles isolated from within cells. Tethering at the 5′ end resulted in the greatest expression of the attached gene sequence and was easily recovered from populations of cells.

Nanoparticle Delivery of Genes In Vivo

FIG. 16 is a schematic representation of magnetic nanoparticle delivery of genes in vivo, as fluorescent micrographs of 5 micron cryosectioned liver from rats injected intrahepatically with nanoparticles tethered to EGFP encoding DNA and coated with lipid. These sections were counterstained with DAPI. Green highlights indicate cells expressing GFP from nanoparticles.

Translation of a gene delivery technology from an in vitro model to in vivo is notoriously difficult. In this disclosure, the inventors were able to transfect hepatocytes in vivo with DNA tethered magnetic nanoparticles. After intrahepatic injection, the animals were maintained for 72 hours prior to sacrifice. The livers were snap frozen, cryosectioned, and counterstained with DAPI. Many GFP positive cells were found within this region, but they occurred in small clusters of 1-10 cells per cluster. Furthermore, there were several clusters of GFP positive cells surrounding the injection site, but overall the transfection efficiency appears somewhat low in this experiment. This may be due to several reasons including a short incubation time and relatively small injection volume of about 100 ul.

The three main findings of these experiments were (i) magnetic nanoparticles can be used for effective gene transfer vectors; (ii) days after gene transfer, the DNA tethered particles could be recovered and the entire gene amplified by PCR; and (iii) DNA tethered magnetic nanoparticles coated with Lipofectamine 2000 could transfer genes in vivo. Cells incubated with these particles showed no visible signs of toxicity (blebbing, apoptosis, etc.), even though the particles are made of iron. The ability to isolate these particles days after their introduction suggests that they are stable within the cellular matrix. This may be due, at least in part, to the conjugation of proteins to the core particle. These proteins could serve as a buffer zone inhibiting contact with fouling agents within the cellular milieu.

EGFP encoding DNA fragments that were transfected into cells showed about half of the fluorescence seen in cells transfected with the circular plasmid. DNA fragments with 5′ or 3′ biotin tags were attached to streptavidin coated magnetic nanoparticles. Free DNA fragments were successfully removed through washing the particles using a magnetic column. Cells exposed to uncoated DNA tethered nanoparticles did not express detectable levels of EGFP. Cells exposed to DNA tethered nanoparticles coated with Lipofectamine 2000 expressed high levels of EGFP. Only cells that expressed EGFP from DNA tethered nanoparticles were recovered with a magnetic column and detected by PCR.

One important finding is that the uncoated DNA tethered magnetic nanoparticles were able to successfully transfect cells in vitro. This result shows that the size range of these particles is, at least, close to optimal since they did not absolutely require the presence of lipofectamine for transfection. Another interesting result is that cells treated with the DNA tethered magnetic nanoparticles coated but also with lipid had expression levels well within range of those transfected with only labeled DNA fragments. These data show that the method can effectively express gene products from DNA tethered to magnetic nanoparticles in either the 5′ or 3′ configuration. Finally, the unlabeled DNA exposed magnetic nanoparticles did not show any appreciable expression of GFP. Therefore the observed expression with the labeled DNA and magnetic nanoparticles was from DNA tethered to magnetic nanoparticles. Tethering appears to greatly increase the expression of these genes by mechanisms not yet well understood.

Experiment 5

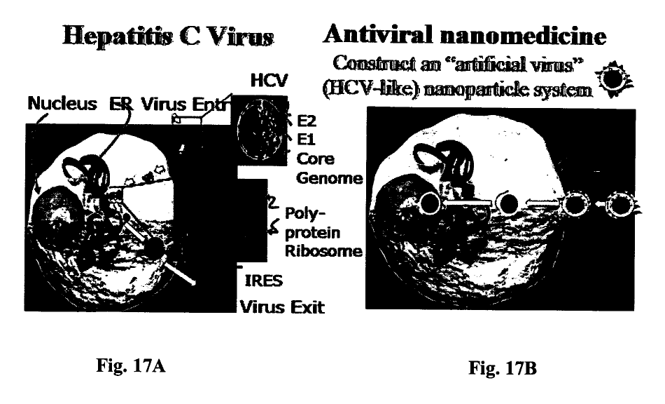

FIG. 17A is a schematic representation of a virus infecting a cell. FIG. 17B is a schematic representation of a nanoparticle construct having a virus appearance with an antiviral payload and will be described in conjunction with FIG. 17A. This experiment mimicked a virus with one layer of the nanoparticle system, but delivered an antiviral molecule from inside the nanoparticle system. In this experiment, a viral biosensor that recognized a hepatitis C viral protein NS3/4 was connected to a ribozyme that cuts the IRES region of the hepatitis C virus (HCV), effectively inactivating it. An E2 protein on HCV helps it target cells to infect liver and other cell types causing organ damage.

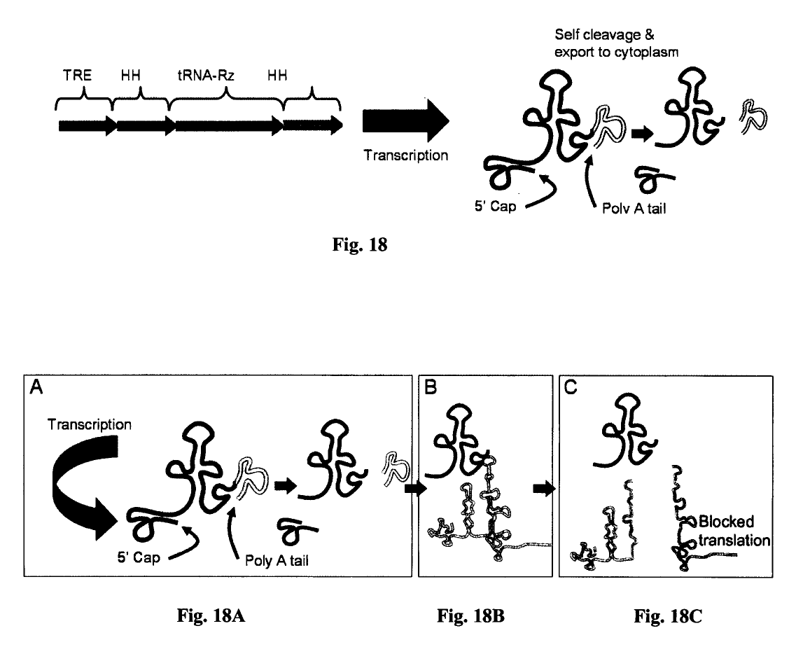

FIG. 18 is a schematic representation of production of a therapeutic gene. FIG. 18A is a schematic representation of transcribed gene being cleaved. FIG. 18B is a schematic representation of the therapeutic gene of FIG. 18A bound to a genome. FIG. 18C is a schematic representation of the internal ribosome entry site (IRES) cleaved by the therapeutic gene of FIG. 18B. The figures will be described in conjunction with each other.

Ribozymes are catalytic, single stranded fragments of RNA that can bind to a complimentary strand of RNA and cleave the complimentary strand at a specific site. Ribozymes can be thought of a targeted molecular scissors that can deactivate targeted RNA strands through cleavage. Earlier efforts improved the ribozyme based approach through splicing a tRNA sequence to the 5′ end of the ribozyme sequence (Kuwabara et al. 2001). This caused the tRNA-ribozyme RNA to be actively exported from the nucleus to the cytoplasm where it can be used to cleave the IRES region of hepatitis C genome Prior efforts also added a tRNA promoter so that the transcription product would have defined ends and be expressed at a high level.

The above system was further developed by the inventors. For a biosensor to activate the expression of the ribozyme, the promoter was a tetracycline response element. Unfortunately, RNA polymerase II is responsible for the transcription of any gene downstream of this promoter and this polymerase caps the 5′ ends of all transcription products. Through other efforts, a self cleaving hammerhead ribozymes were developed for use on the 5′ and 3′ ends of the tRNA-ribozyme construct, as shown in FIG. 18. This construct is exemplary and other constructs are certainly possible. These flanking ribozymes should self cleave once expressed and result in a tRNA-ribozyme construct with defined ends, as shown in FIGS. 18, 18A. The purpose of the defined ends and tRNA regions of this construct are nuclear export. The stem loop region of the tRNA is responsible for its active transport out of the nucleus. Coincidentally, the target of this ribozyme based system is one of the stem loops found in the hepatitis C virus genome. The 5′ non-translated region of the viral mRNA genome forms the IRES and primarily promotes translation of the viral genome, shown in FIG. 18B. The 5′ non-translated region including the part encoding the IRES is also thought to contribute to viral replication. By cleaving the IRES at this particular point, shown in FIG. 18C, test results show a significant decrease in IRES mediated activity and in hepatitis C virus replication.

FIGS. 19A-19D are a representation of a single cell ribozyme therapy, showing the results with control data. FIG. 19A represents cells with stained DNA. FIG. 19B represents cells stained with a molecular biosensor. FIG. 19C represents a therapeutic response. FIG. 19D represents a composite image. The figures will be described in conjunction with each other. The progression of FIGS. 19A-19D show the reduction of NSE HCV viral protein under the action of ribozyme therapy.

To demonstrate the feasibility of combating viral infection at the single cell level using nanomedicine, the inventors applied ribozyme therapy to HCV infected single cells. In a model cell system in-vitro, a nanomedical system was constructed with a ribozyme directed against the IRES version of the hepatitis C virus (HCV). The cells shown in FIGS. 19A-19D are both infected with HCV, but only cell # 2 on the right portion of each figure has been treated with the ribozyme. In FIG. 19A, cell #1 (untreated) and cell # 2 (treated) are stained for their DNA. In FIG. 19B, the cells are stained with a molecular biosensor directed against the ribozyme. Cell # 1 is untreated, and cell # 2 is ribozyme-treated as seen by the ribozyme biosensor present in cell # 2, but absent in cell # 1. The therapeutic response of the ribozyme is shown in FIG. 19C which shows a continued presence of NSE protein in untreated cell # 1 but its reduction in ribozyme-treated cell # 2. FIG. 19D represents a composite image.

While the foregoing is directed to various embodiments of the present invention, other and further embodiments may be devised without departing from the basic scope thereof. Other embodiments within the scope of the claims herein will be apparent to one skilled in the art from consideration of the specification and practice of the invention as disclosed herein. For example, other types virus or mimicking molecules can be used, different sequences of interaction with the cell and intracellular components can be arranged, other testing techniques can verify the results, and other variations, given the fundamentals of the disclosure herein. It is intended that the specification, together with the example, be considered exemplary only, with the scope and spirit of the invention being indicated by the claims which follow.

The various methods and embodiments of the invention can be included in combination with each other to produce variations of the disclosed methods and embodiments, as would be understood by those with ordinary skill in the art, given the understanding provided herein. Also, various aspects of the embodiments could be used in conjunction with each other to accomplish the understood goals of the invention. Also, the directions such as “top,” “bottom,” “left,” “right,” “upper,” “lower,” and other directions and orientations are described herein for clarity in reference to the figures and are not to be limiting of the actual device or system or use of the device or system. Unless the context requires otherwise, the word “comprise” or variations such as “comprises” or “comprising”, should be understood to imply the inclusion of at least the stated element or step or group of elements or steps or equivalents thereof, and not the exclusion of a greater numerical quantity or any other element or step or group of elements or steps or equivalents thereof. The device or system may be used in a number of directions and orientations. Further, the order of steps can occur in a variety of sequences unless otherwise specifically limited. The various steps described herein can be combined with other steps, interlineated with the stated steps, and/or split into multiple steps. Additionally, the headings herein are for the convenience of the reader and are not intended to limit the scope of the invention.

Further, any references mentioned in the application for this patent as well as all references listed in the information disclosure originally filed with the application are hereby incorporated by reference in their entirety to the extent such may be deemed essential to support the enabling of the invention. However, to the extent statements might be considered inconsistent with the patenting of the invention, such statements are expressly not meant to be considered as made by the Applicants.

### REFERENCES

Ai, H., Jones, S. A., Lvov, Y. M., “Biomedical applications of electrostatic layer-by-layer nano-assembly of polymers, enzymes, and nanoparticles.” Cell Biochem Biophys, 39, 2003.
Albig, A. (2001) Isolation of mRNA binding proteins using the μMACS™ Streptavidin Kit. MACS&more, 5, 6-7.
Bearman, G. H., Frasier, S. E., Lanford, R. D. (2002) System and method for monitoring cellular activity; U.S. Pat. No. 6,403,328.
Benns, J. M. and Kim, S. W. (2000) Tailoring new gene delivery designs for specific targets. J Drug Target, 8, 1-12.
Bonnemain, B. (1998) Superparamagnetic agents in magnetic resonance imaging: physicochemical characteristics and clinical applications. A review. J Drug Target, 6, 167-174.
Campbell, N. A. and Reece, J. B., Biology, Benjamin Cummings, San Francisco, 2002.
Chan, W. C., D. J. Maxwell, et al. (2002) Luminescent quantum dots for multiplexed biological detection and imaging. Curr Opin Biotechnol 13(1): 40-6.
De Smedt, S. C., J. Demeester, et al. (2000) Cationic polymer based gene delivery systems. Pharm Res 17(2): 113-26.
Douglas, S. J., Davis, S. S. and Illum, L. (1987) Nanoparticles in drug delivery. Crit Rev Ther Drug Carrier Syst, 3, 233-261.
Dubertret, B., P. Skourides, et al. (2002) In vivo imaging of quantum dots encapsulated in phospholipid micelles. Science 298(5599): 1759-62.
Hogemann, D., Ntziachristos, V., Josephson, L. and Weissleder, R. (2002) High throughput magnetic resonance imaging for evaluating targeted nanoparticle probes. Bioconjug Chem, 13, 116-121.
Jaiswal, J. K., H. Mattoussi, et al. (2003) Long-term multiple color imaging of live cells using quantum dot bioconjugates. Nat Biotechnol 21(1): 47-51.
Koropchak, J. A., Sadain, S., Yang, X., Magnusson, L. E., Heybroek, M., Anisimov, M. and Kaufman, S. L. (1999) Nanoparticle detection technology for chemical analysis. Anal Chem, 71, 386A-394A.
Kreuter, J., Tauber, U. and Illi, V. (1979) Distribution and elimination of poly(methyl-2-14C-methacrylate) nanoparticle radioactivity after injection in rats and mice. J Pharm Sci, 68, 1443-1447.
Krotz, F., Sohn, H. Y., Gloe, T., Plank, C. and Pohl, U. (2003) Magnetofection Potentiates Gene Delivery to Cultured Endothelial Cells. J Vasc Res, 40, 425-434.
Krotz, F., Wit, C., Sohn, H. Y., Zahler, S., Gloe, T., Pohl, U. and Plank, C. (2003) Magnetofection-A highly efficient tool for antisense oligonucleotide delivery in vitro and in vivo. Mol Ther, 7, 700-710.
Kuwabara, T., M. Warashina, S. Koseki, M. Sano, J. Oikawa, K. Nakayama, and K. Taira. (2001). Significantly higher activity of a cytoplasmic hammerhead ribozyme than a corresponding nuclear counterpart: Engineered trnas with an extended 3′ end can be exported efficiently and specifically to the cytoplasm in mammalian cells. Nucleic Acids Res 29, no. 13: 2780-8.
Lanza, G. M., Yu, X., Winter, P. M., Abendschein, D. R., Karukstis, K. K., Scott, M. J., Chinen, L. K., Fuhrhop, R. W., Scherrer, D. E. and Wickline, S. A. (2002) Targeted antiproliferative drug delivery to vascular smooth muscle cells with a magnetic resonance imaging nanoparticle contrast agent: implications for rational therapy of restenosis. Circulation, 106, 2842-2847.
Leary, J. F. (1994) Strategies for rare cell detection and isolation. Methods Cell Biol 42 Pt B: 331-358.
Lvov, Y. and F. Caruso (2001) Biocolloids with ordered urease multilayer shells as enzymatic reactors. Anal Chem 73(17): 4212-7.
Lvov, Y., A. A. Antipov, et al. (2001) Urease Encapsulation in Nanoorganized Microshells. Nano Letters 1(3): 125-128.
Mamedov, A. A., A. Belov, M. Giersig, N. N. Mamedova, and N. A. Kotov. (2001) Nanorainbows: Graded semiconductor films from quantum dots. J Am Chem Soc 123, no. 31: 7738-7739.
Mitchell, D. G. (1997) MR imaging contrast agents—what's in a name? J Magn Reson Imaging, 7, 1-4.
Mizutani, Y., Y. Okada, O. Yoshida, M. Fukumoto, and B. Bonavida. Doxorubicin sensitizes human bladder carcinoma cells to fas-mediated cytotoxicity. Cancer 79, no. 6: 1180-1189, 1997.
Mullikin, J. C. (1993) In Press, D. U. (ed.), Discrete and Continuous Methods for Three-Dimensional Image Analysis, pp. 47-48.
Ohki, E. C., Tilkins, M. L., Ciccarone, V. C. and Price, P. J. (2001) Improving the transfection efficiency of post-mitotic neurons. J Neurosci Methods, 112, 95-99.
Panyam, J. and Labhasetwar, V. (2003) Biodegradable nanoparticles for drug and gene delivery to cells and tissue. Adv Drug Deliv Rev, 55, 329-347.
Petersein, J., Saini, S. and Weissleder, R. (1996) Liver. II: Iron oxide-based reticuloendothelial contrast agents for MR imaging. Clinical review. Magn Reson Imaging Clin N Am, 4, 53-60.
Plank, C., Anton, M., Rudolph, C., Rosenecker, J. and Krotz, F. (2003) Enhancing and targeting nucleic acid delivery by magnetic force. Expert Opin Biol Ther, 3, 745-758.
Plank, C., Scherer, F., Schillinger, U., Bergemann, C. and Anton, M. (2003) Magnetofection: enhancing and targeting gene delivery with superparamagnetic nanoparticles and magnetic fields. J Liposome Res, 13, 29-32.
Plank, C., Schillinger, U., Scherer, F., Bergemann, C., Remy, J. S., Krotz, F., Anton, M., Lausier, J. and Rosenecker, J. (2003) The magnetofection method: using magnetic force to enhance gene delivery. Biol Chem, 384, 737-747.
Sandberg, J. A., C. D. Sproul, K. S. Blanchard, L. Bellon, D. Sweedler, J. A. Powell, F. A. Caputo, D. J. Kornbrust, V. P. Parker, T. J. Parry, and L. M. Blatt. (2000). Acute toxicology and pharmacokinetic assessment of a ribozyme (angiozyme) targeting vascular endothelial growth factor receptor mRNA in the cynomolgus monkey. Antisense Nucleic Acid Drug Dev 10(3): 153-62.
Scherer, F., Anton, M., Schillinger, U., Henke, J., Bergemann, C., Kruger, A., Gansbacher, B. and Plank, C. (2002) Magnetofection: enhancing and targeting gene delivery by magnetic force in vitro and in vivo. Gene Ther, 9, 102-109.
Sheard, M. A. (2001) Ionizing radiation as a response-enhancing agent for CD95-mediated apoptosis. Int. J. Cancer 96: 213-220.
Stewart, M., R. P. Baker, et al. (2001) Molecular mechanism of translocation through nuclear pore complexes during nuclear protein import. FEBS Lett 498(2-3): 145-9.
Sun, M. L. and Zhang, H. (2001) [The development of nanoparticles on DNA isolation and purification]. Sheng Wu Gong Cheng Xue Bao, 17, 601-603.
Tice R R, Agurell E, Anderson D, Burlinson B, Hartmann A, Kobayashi H, Miyamae Y, Rojas E, Ryu J C, Sasaki Y F. (2000) Single cell gel/comet assay: guidelines for in vitro and in vivo genetic toxicology testing. Environ Mol Mutagen 35(3):206-221.
Vijayanathan, V., Thomas, T. and Thomas, T. J. (2002) DNA nanoparticles and development of DNA delivery vehicles for gene therapy. Biochemistry, 41, 14085-14094.
Violante, M. R. (1990) Potential of microparticles for diagnostic tracer imaging. Acta Radiol Suppl, 374, 153-156.
Wang, Y. X., Hussain, S. M. and Krestin, G. P. (2001) Superparamagnetic iron oxide contrast agents: physicochemical characteristics and applications in MR imaging. Eur Radiol, 11, 2319-2331.
Winter, P. M., Caruthers, S. D., Kassner, A., Harris, T. D., Chinen, L. K., Allen, J. S., Lacy, E. K., Zhang, H., Robertson, J. D., Wickline, S. A. et al. (2003) Molecular imaging of angiogenesis in nascent Vx-2 rabbit tumors using a novel alpha(nu)beta3-targeted nanoparticle and 1.5 tesla magnetic resonance imaging. Cancer Res, 63, 5838-5843.
Zauner, W., N. A. Farrow, et al. (2001) In vitro uptake of polystyrene microspheres: effect of particle size, cell line and cell density. J Control Release 71(1): 39-51.
Zeta-Meter, I. (1997) Zeta-Potential: A Complete Course in 5 Minutes, Zeta-Meter, Inc., Staunton.
Zhu, M. and Fahl, W. E. (2000) “Development of a green fluorescent protein microplate assay for the screening of chemopreventive agents”, Anal Biochem, 287(2), 210-7.

1. A multi-functional and multi-step nanodelivery system having one or more nanoparticles, the nanoparticles comprising:
a. one or more targeting molecules adapted to target the nanoparticle to one or more cells;
b. one or more cellular entry facilitating molecules coupled to the targeting molecules;
c. one or more anchoring molecules coupled to the entry facilitating molecules;
d. one or more drugs or genes coupled to the anchoring molecules; and
e. one or more molecular biosensors adapted to control an intracellular delivery of a quantity of the drugs or genes in a feedback loop at a single cell level.

2. The system of claim 1, further comprising intracellular re-targeting molecules coupled to the entry facilitating molecules.

3. The system of claim 1, wherein the molecular biosensors provide one or more molecular biosensor feedback loops for controlled delivery.

4. The system of claim 1, wherein the targeting molecules comprise an antibody, a DNA, a RNA, a peptide sequence, a ligand, a thioaptamer, or a combination thereof.

5. The system of claim 1, wherein the targeting molecules are adapted to target a desired location with Boolean logic sequences of positive and negative identification.

6. The system of claim 1, wherein the targeting molecules comprise virus-associated or mimicking molecules.

7. The system of claim 6, wherein the drug or gene comprises an antiviral drug or gene.

8. The system of claim 1, wherein the nanoparticle is adapted to produce additional quantities of the drug or gene from an intracellular location.

9. The system of claim 1, wherein additional quantities of the drug or gene are obtained by interaction with one or more targeted intracellular organelle.

10. The system of claim 9, wherein the intracellular organelle comprise a nucleus, a mitochondria, an endoplasmic reticulum, other subcellular parts or regions of the cell, or a combination thereof.

11. The system of claim 1, wherein the molecular biosensor is disposed in the nanodelivery system such that it switches on and off production of quantities of the drug or gene based in the feedback loop at the single cell level.

12. The system of claim 1, wherein the biosensor is adapted to periodically check for an expressed gene and provide the drug or gene based upon an occurrence of the expressed gene.

13. The system of claim 12, wherein the biosensor is adapted to stop the provision of the drug or gene when the expressed gene is inactive.

14. The system of claim 1, wherein the biosensor is adapted to sense intracellular stress-related molecules causing damage to a cell comprising reactive oxygen species proteins generated during radiation, chemical interaction with the cell, or pathogen-induced interaction with the cell, or a combination thereof, and to control the delivery of the drug or gene to at least partially repair the damaged cell.

15. The system of claim 1, further comprising a signaling molecule released upon a reaction of the biosensor and a target molecule.

16. The system of claim 15, further comprising a reporter molecule generated by the signaling molecule.

17. The system of claim 1, wherein the molecular biosensor comprises a multi-fusion protein molecular biosensor that is adapted to locate a target cell or cellular compartment, detect a presence of the target molecule based on the specific reaction between the target molecule and the biosensor, and release a signaling molecule that in turn generates a reporter molecule.

18. The system of claim 1, wherein one or more nanoparticles are coated to reduce cytotoxicity.

19. The system of claim 1, wherein one or more nanoparticles comprise a magnetic nanoparticle.

20. The system of claim 19, wherein the nanodelivery system is adapted to extract at least a portion of the nanoparticles from the cell after cellular entry.

21. The system of claim 1, wherein one or more nanoparticles comprise a semiconductor material core.

22. A nanodelivery system having two or more nanoparticles for delivery of a drug or gene to an intracellular location, comprising:
a. a first nanoparticle having a first targeting molecule and a first component of a drug or gene;
b. a second nanoparticle having a second targeting molecule and a second component of a drug or gene different than the first component;
the drug or gene being adapted to provide a desired reaction upon a successful targeting of the first and second targeting molecules and a combination of the first and second components of the drug or gene.

23. The system of claim 22, further comprising a third targeting molecule, adapted to not bind to a cell of interest, coupled to at least one of the nanoparticles and adapted to at least partially block the reaction between the first and second components upon a successful targeting by the third targeting molecule.

24. The system of claim 23, wherein the third targeting molecule inhibits treatment of non-desired, bystander cells.

25. The system of claim 22, further comprising a third nanoparticle having a third targeting molecule adapted to at least partially block the reaction between the first and second components upon a successful targeting of the third targeting molecule.

26. The system of claim 22, further comprising:
a. one or more cellular entry facilitating molecules coupled to the targeting molecules;
b. one or more anchoring molecules coupled to the entry facilitating molecules;
c. one or more molecular biosensors adapted to control an intracellular delivery of a quantity of the drugs or genes.

27. A multi-functional and multi-step nanodelivery system having one or more nanoparticles, the nanoparticles comprising:
a. one or more targeting molecules adapted to target the nanoparticle to one or more cells; and
b. one or more cellular entry facilitating molecules coupled to the targeting molecules;
the nanoparticle being adapted to manufacture quantities of the desired drug or gene from an intracellular location of the cell using one or more intracellular native components.

28. The system of claim 27, further comprising one or more molecular biosensors adapted to control the intracellular manufacture of the drugs or genes.

29. The system of claim 27, wherein additional quantities of the drug or gene are obtained by interaction with one or more targeted intracellular organelle.

30. The system of claim 29, wherein the intracellular organelle comprise a nucleus, a mitochondria, an endoplasmic reticulum, other subcellular parts or regions of the cell, or a combination thereof.

31. A multi-functional and multi-step nanodelivery system having one or more nanoparticles, the nanoparticles comprising:
a. one or more targeting molecules adapted to target the nanoparticle to one or more cells; and
b. one or more cellular entry facilitating molecules coupled to the targeting molecules; and
c. one or more anchoring molecules coupled to the entry facilitating molecules and adapted to locate at least one nanoparticle at an intracellular selected site for subsequent intracellular drug or gene delivery.

32. A process for producing a multi-functional and multi-step nanodelivery system, acting in an autonomous controlled sequence of events at the molecular level, comprising:
a. obtaining a nanoparticle;
b. coupling a drug or gene to the nanoparticle;
c. coupling a molecular biosensor to the drug or gene; and
d. coupling a cell targeting molecule to the molecular biosensor.

33. The process of claim 32, further comprising coupling a cellular entry facilitating molecule to the targeting molecule.

34. The process of claim 33, further comprising coupling an anchoring molecule to the cellular entry facilitating molecule.

35. The process of claim 33, further comprising coupling an intracellular target molecule to the targeting molecule.

36. The process of claim 32, further comprising coating the nanoparticle with a biocompatible coating.

37. The process of claim 32, further comprising locating the biosensor in the nanodelivery system to control a delivery of the drug or gene.

38. The process of claim 32, further comprising introducing the nanodelivery system into a living biological system and allowing the nanodelivery system to enter a desired cell and perform a repair on an intracellular component of the cell.

39. The process of claim 38, wherein the repair comprises cleaving a virus in the cell to inactivate the virus.

40. The process of claim 38, wherein the repair comprises repairing a radiation damaged portion of the cell.

41. The process of claim 40, further comprising checking for a radiation damaged portion of the cell.
